(19) United States
(12) Patent Application Publication
(10) Pub. No.: US 2024/0093177 A1
(43) Pub. Date: Mar. 21, 2024

(54) METHODS TO SELECT ANTIBODIES SPECIFIC TO COMPLEX MEMBRANE ANTIGENS

(71) Applicant: VACCINEX, INC., Rochester, NY (US)

(72) Inventors: Malgorzata Gil-Moore, Rochester, NY (US); Maria G.M Scrivens, Pittsford, NY (US); Renee A. Kirk, Rochester, NY (US); Leslie A. Balch, Rochester, NY (US); Loretta L. Mueller, Rochester, NY (US); Ernest S. Smith, Rochester, NY (US)

(21) Appl. No.: 18/333,339

(22) Filed: Jun. 12, 2023

Related U.S. Application Data
(60) Provisional application No. 63/350,939, filed on Jun. 10, 2022.

(51) Int. Cl. C12N 15/10; C07K 16/28

(52) U.S. Cl. CPC C12N 15/1044; C07K 16/2887; C07K 16/2896; C12N 15/1013; C12N 15/1065

(57) ABSTRACT

This disclosure provides compositions and methods for high throughput screening, selecting, and identifying antibodies or antibody-like molecules that bind to a target integral membrane protein.

### CROSS-REFERENCE TO RELATED APPLICATION

This is a non-provisional of pending U.S. provisional application Ser. No. 63/350,939, filed Jun. 10, 2023, the entirety of which application is incorporated by reference herein.

### SEQUENCE LISTING

The instant application contains a Sequence Listing which has been submitted electronically in XML format and is hereby incorporated by reference in its entirety. Said XML copy, created on Jun. 12, 2023, is named 8555_042_SL.xml and is 61,440 bytes in size.

### BACKGROUND

Many important targets for therapeutic antibodies are integral membrane proteins (IMPs), e.g., multi-pass membrane proteins (GPCRs, Ion Channels, etc.) that are difficult to express and purify in a conformationally-intact state. The absence of properly folded target proteins in an isolated state makes the identification and selection of antibodies to these targets challenging. While certain IMPS can be expressed on the surface of cells, e.g., mammalian cells, whole cells are problematic for use in antibody discovery because they are complex antigen mixtures, target expression can be low, and because certain display packages used to construct antibody libraries (e.g., vaccinia virus antibody libraries) can bind to whole cells non-specifically. Screening antibodies against a desired antigen, within a selected cell line, may be difficult due to the abundance of irrelevant organic molecules, which can potentially obscure the antigen of interest.

Integral membrane protein display on poxvirus extracellular enveloped virions, e.g., vaccinia virus, as disclosed in U.S. Pat. No. 10,577,427, which is incorporated herein in its entirety by reference, provides a method for expressing and displaying target IMPS of interest in their native conformation at a sufficient concentration and with minimal competition from other cell proteins to allow for identification and selection of therapeutic antibodies and antibody-like molecules. However, both in vitro and in vivo antibody screening methods are laborious, costly and inefficient. For example, monoclonal antibodies (Mabs) are often selected from antigen-specific single B cells derived from different hosts, which are notably short-lived in ex vivo culture conditions and hence, arduous to interrogate. Alternatively, Mabs can be generated independently of antigen-specific B cells, using display technologies. While cell sorting offers the ability to interrogate a large library pool or B cell repertoire, the technique requires soluble antigen. Thus, there remains a need for efficient high throughput antibody screening methods for selecting and identifying antibodies to IMPs.

### SUMMARY

This disclosure provides compositions and methods for high throughput screening, selecting, and identifying of antibodies or antibody-like molecules that bind to a target IMP of interest.

In one aspect, the disclosure provides a method to select binding molecules that bind to a target integral membrane protein (IMP) comprising:
(a) attaching an antigen virion comprising a pox virus that comprises the integral membrane protein (IMP) or a fragment thereof fused with a poxvirus extracellular enveloped virion (EEV) protein or a fragment thereof to form an IMP-EEV fusion protein, wherein the poxvirus expresses the target IMP or fragment thereof in native conformation as part of the outer envelope membrane of the EEV to a solid support to form a coupled antigen virion, wherein said IMP or fragment thereof comprises at least one extra-membrane region, at least one transmembrane region, and at least one intra-membrane region;
(b) contacting said coupled antigen virion with an antibody display library, wherein the library comprises display packages displaying a plurality of antigen binding domains;
(c) extracting antibody variable genes or fragments thereof from display packages that bind to the coupled antigen virion and cloning the variable light chain (VL) gene or fragment thereof and variable heavy chain (VH) gene or fragment thereof from said display package into a plasmid vector in frame with a polynucleotide sequence encoding a pox virus anchor protein, e.g., an extra-cellular enveloped virion protein, or functional fragment thereof such that the VL, VH and sequence encoding the poxvirus anchor protein are co-expressed as a single polypeptide;
(d) transfecting mammalian cells with the plasmid vector of step (c) such that transfected cells express VL and VH antigen binding domains on the mammalian cell surface;
(e) screening the transfected cells with the antigen virion coupled to a detectable solid support; and
(f) recovering cells that display an antigen binding domain specific for the target.

In certain embodiments the solid support in step (a) is streptavidin labelled magnetic beads. In other embodiments, the poxvirus is fowlpox virus and the biotin label is a biotin-anti-fowlpox antibody. In other embodiments the poxvirus is a biotinylated vaccinia virus Ankara (MVA). In some embodiments the poxvirus anchor protein is the vaccinia virus A56R protein. In some embodiments the mammalian cells are CHO cells. In some embodiments the IMP is a multi-pass IMP such as an ion channel or a G protein. The multi-pass IMP can have either an even number or odd number of transmembrane domains.

In another aspect the disclosure provides a method to select binding molecules that bind to a target integral membrane protein (IMP) comprising:
(a) isolating plasma cells from an animal, such as a mammal, shark, or chicken, immunized with an antigen comprising the target integral membrane protein (IMP) or a fragment thereof;
(b) seeding the plasma cells into pools comprising a plurality of plasma cells and growing them in nutrient media to a desired cell density;
(d) performing one or more assays of the plasma cells to identify cells that express binding molecules that bind to the target IMP protein; and
(e) recovering the plasma cells that express binding molecules that bind to the target IMP protein.

In certain embodiments, isolated plasma cells are seeded into pools containing a plurality of the plasma cells, e.g., of 1000 cells or less, such as 100 plasma cells or less. In some embodiments of this aspect, a first ELISA is performed to identify cells that express binding molecules that bind to the target IMP protein, followed by at least one further ELISA assay in which the cells that are identified by the first ELISA assay are diluted prior to performing the further ELISA assay to identify cells that express binding molecules that bind to the target IMP protein. In some embodiments the cells that are recovered are used to generate an antibody display library, wherein the library comprises display packages displaying a plurality of antigen binding domains. In certain embodiments, the animal is immunized with an antigen virion comprising a pox virus that expresses the target IMP in native conformation as part of the outer envelope membrane of the EEV.

In another aspect of the disclosure, there is provided a method to select binding molecules that specifically bind to a target integral membrane protein (IMP) comprising:
(a) isolating B cells from an animal, such as a mammal, shark, chicken, immunized with the target integral membrane protein (IMP) or fragment thereof;
(b) sorting the B cells to isolate antigen-specific B cells that express IgG that specifically binds target IMP; and
(c) performing single cell analysis to identify the Immunoglobulin variable region genes expressed by the sorted B cells.

In some embodiments of this aspect, the single cell analysis comprises RT-PCR. In some embodiments of this aspect, the method further comprises isolating and cloning variable heavy chain genes and/or variable light chain genes from individual sorted B cells. In some embodiments of this aspect of the disclosure, the B cells are sorted with target IMP coupled to a detectable solid support, such as a streptavidin fluorescent bead. In certain embodiments, a phage Fab display library is generated from variable heavy chain (VH) and variable light chain (VL) cDNAs generated from RNA isolated from the antigen-specific B cells that express IgG that binds target IMP. In certain embodiments, the phage Fab display library is panned to eliminate anti-poxvirus binding molecules and enrich for anti-target IMP binding molecules. In other embodiments, the method includes a further step of isolating the VH and VL genes (V genes) from the phage Fab display library and subcloning the V genes into an expression vector, such as a mammalian expression vector, while maintaining VH and VL pairing that was present in individual phage as a mini-library (ML). In certain embodiments of this aspect, the IMP is a multi-pass IMP. In this and every aspect of the disclosure, the multi-pass IMP can have either an even number or odd number of transmembrane domains.

In another aspect of the disclosure, there is provided a library made by the methods disclosed herein.

In another aspect of the disclosure, antibodies that specifically bind to CD20 and which are defined by their VH and VL chain sequences are disclosed.

### BRIEF DESCRIPTION OF THE DRAWINGS

FIGS. 1A-1D show flow cytometry dot plots for antigen specific binding for anti-CD20 or CD39 antibody expressing CHO cells following staining with CD20- or CD39-expressing EEV bound to fluorescent yellow (FIGS. 1A and 1B, left panels) versus blue beads (FIGS. 1C and 1D, right panels) with biotin. The cells expressing antibody and binding FPV-coated beads were identified as double positive events.

FIGS. 2A-2B show flow cytometry dot plots for antigen specific binding for anti-SEMA4D antibody expressing CHO cells following staining with SEMA4D-expressing MVA bound to fluorescent yellow (FIG. 2A, left panels) versus blue beads (FIG. 2 B, right panels) with biotin. The cells expressing antibody and binding MVA-coated beads were identified as double positive events.

FIGS. 3A-3C show antigen specific binding using CHO cells stably expressing anti-SEMA4D (FIG. 3A) or anti-SEMA4D antibody library (FIG. 3B) derived by phage panning.

FIG. 3C shows the sort gate for the cells that were sorted.

FIGS. 4A-4C show antigen virus sorting strategies. Balb/c mice were immunized with EEV MVA-T7-Cd20-G-F, B cells were isolated and stained with anti-B220, anti-IgM, anti-IgG1/IgG2ab and FPV-CD20 (positive) or FPV-CD39 (negative) (FIG. 4A). The B cells were gated on B220+/IgM−/IgG1,2ab+(FIG. 4B: FPV-CD20 and 4C: FPV-CD39).

FIG. 5A shows a plasma cell screening strategy of the disclosure. Mice were immunized with MVA-CD20. Plasma cells were isolated and cultured at 100 cells/well in 96 well plate for 3 days. The cell culture supernatant was then tested by ELISA on wells coated with FPV-CXCR4 or wild type FPV. FIG. 5B shows individual histograms for CD20 positive antibodies selected from a phage display library created from plasma cells isolated from mice immunized with an MVA-CD20 construct of the disclosure.

FIG. 6 is a drawing of vector EFGDV3-Mab67-A56R (CD100).

FIG. 7 is a drawing of vector EFDGV3-Mab15735-A56R (CD20).

FIG. 8 is a drawing of vector EFDGV3-Mab26089-A56R (CD39).

### DETAILED DESCRIPTION

This disclosure provides methods and compositions for high throughput screening, selecting, and identifying of antibodies or antibody-like molecules that bind to target integral membrane proteins (IMPs), e.g., multi-pass IMPS or fragments thereof.

### Definitions

The term “a” or “an” entity refers to one or more of that entity; for example, “a binding molecule,” is understood to represent one or more binding molecules. As such, the terms “a” (or “an”), “one or more,” and “at least one” can be used interchangeably herein.

The term, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides that do not possess a defined three-dimensional structure, but rather can adopt a large number of different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides that have been separated, fractionated, or partially or substantially purified by any suitable technique.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides that retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

As used herein the term “integral membrane protein” or “IMP” refers to a protein or polypeptide that is attached to a biological membrane. One example of an IMP is a transmembrane protein, which spans the lipid bilayer of the biological membrane one or more times. Single-pass membrane proteins cross the membrane only once, while multi-pass membrane proteins weave in and out, crossing several times. Type I single-pass proteins are positioned with their amino terminus on the outer side of the membrane or “extra-membrane” and their carboxyl-terminus on the interior side of the membrane, or “intra-membrane.” Type II single-pass proteins have their amino-terminus on the intra-membrane side. Multi-pass transmembrane proteins pass through the membrane two or more times and can have a variety of different topologies. Those proteins with an even number of transmembrane domains will have both their amino terminus and carboxy terminus on the same side of the membrane. One example of such a protein is CD20, which is expressed on B cells. Those with an odd number of transmembrane domains will have their amino- and carboxy termini on opposite sides of the membrane. Examples include G-protein coupled receptors, which typically have 7 transmembrane domains, with the amino terminus on the extra-membrane side and the carboxy terminus on the intra-membrane side. Certain IMPS do not have transmembrane domains and are instead anchored to the membrane, e.g., via a lipid such as glycosylphosphatidylinositol or palmitoyl group. IMPs have myriad biological functions including, but not limited to transporters, linkers, channels, receptors, enzymes, energy transduction or cell adhesion.

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, that has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “non-naturally occurring” polynucleotide, or any grammatical variants thereof, is a conditional definition that explicitly excludes, but only excludes, those forms of the polynucleotide that are well-understood by persons of ordinary skill in the art as being “naturally-occurring,” or that are, or that might be at any time, determined or interpreted by a judge or an administrative or judicial body to be, “naturally-occurring.”

As used herein, a “coding region” is a portion of nucleic acid that consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide normally can include a promoter and/or other transcription or translation control elements operably associated with one or more coding regions. An operable association occurs when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit .beta.-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Poxvirus promoters (e.g. p7.5 or H5) or the bacteriophage T7 promoter can also be used as transcription control regions. When employing a T7 promoter, an inducible vaccinia expression system can be utilized. The vaccinia expression system can include, but is not limited, to a first recombinant vaccinia virus that encodes the entire bacteriophage T7 gene 1 coding region for T7 RNA polymerase, and a second recombinant vaccinia virus that encodes a gene of interest flanked by a T7 promoter and termination regulatory elements. Dual infection of eukaryotic cells with both recombinant vaccinia viruses results in synthesis of the T7 RNA polymerase and expression of the gene of interest controlled by the T7 promoter.

Similarly, a variety of translation control elements are known to those of ordinary skill in the art. These include, but are not limited to ribosome binding sites, translation initiation and termination codons, and elements derived from picornaviruses (particularly an internal ribosome entry site, or, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions that encode secretory or signal peptides, which direct the secretion of a polypeptide encoded by a polynucleotide as disclosed herein. According to the signal hypothesis, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, a “library” is a representative genus of polynucleotides, e.g., a group of polynucleotides related through, for example, their origin from a single animal species, tissue type, organ, or cell type, where the library collectively comprises at least two different species within a given genus of polynucleotides. A library of polynucleotides can include, e.g., at least two, at least 5, at least 10, 100, 103, 104, 105, 106, 107, 108, or 109 different species within a given genus of polynucleotides. In certain aspects, a library of polynucleotides as provided herein can encode a plurality of polypeptides that contains a polypeptide of interest. In certain aspects, a library of polynucleotides as provided herein can encode a plurality of immunoglobulin subunit polypeptides, e.g., heavy chain subunit polypeptides or light chain subunit polypeptides. In this context, a “library” as provided herein comprises polynucleotides of a common genus, the genus being polynucleotides encoding immunoglobulin subunit polypeptides of a certain type and class e.g., a library might encode a human μ, [symbol] -1, [symbol] -2, [symbol] -3, [symbol] -4, α1, α-2, epsilon, or delta heavy chain, or a human K or λ light chain. Although each member of any one library constructed according to the methods provided herein can encode the same heavy or light chain constant region and/or a membrane anchoring domain, the library can collectively comprise at least two, at least 5, or at least 10, 100, 103, 104, 105, 106, 107, 108, or 109 different variable region associated with the common constant region.

In other embodiments, the library can be a plurality of immunoglobulin single-chain fragments that comprise a variable region, such as a light chain variable region or a heavy chain variable region, and/or both a light chain variable region and a heavy chain variable region, e.g., an ScFv fragment.

As used herein, a “display library” is a library of polynucleotides each carried in a “display package” that expresses the polypeptide encoded by the library polynucleotide on its surface. An antibody display library, for example, can include a plurality of display packages, each displaying an antigen binding domain of an antibody on its surface. When the display library is permitted to interact with an antigen of interest, e.g., immobilized on a solid surface, those display packages that bind the antigen can be isolated from the rest of the library and recovered. The polynucleotide encoding the antigen binding domain displayed on the surface of the display package can then be isolated. Display libraries include, without limitation, phage display libraries in bacteria or libraries in eukaryotic systems, e.g., yeast display, mammalian cell display, e.g., CHO cells, retroviral display, or expression in DNA viruses such as poxviruses. See, e.g., U.S. Pat. No. 7,858,559, and U.S. Patent Appl. Publication No. 2013-028892, which are incorporated herein by reference in their entireties. In certain aspects, an antibody display library can be prepared in a poxvirus, e.g., vaccinia virus vector, as fusion proteins with an EEV-specific protein, such that the “display packages” are EEV particles. See U.S. Patent Appl. Publication No. 2013-028892.

Such display libraries can be screened against the IMP fusion proteins displayed on the surface of EEV as provided herein.

By “recipient cell” or “host cell” or “cell” is meant a cell or population of cells in which a recombinant protein can be expressed, a virus can be propagated, or polynucleotide libraries as provided herein can be constructed and/or propagated. A host cell as provided herein is typically a eukaryotic cell or cell line, e.g., a vertebrate, mammalian, rodent, mouse, primate, or human cell or cell line. By “a population of host cells” is meant a group of cultured cells which a “library” as provided herein can be constructed, propagated, and/or expressed. Any host cell which is permissive for vaccinia virus infectivity or fowl pox virus infectivity is suitable for the methods provided by this disclosure. Host cells for use in the methods provided herein can be adherent, e.g., host cells that grow attached to a solid substrate, or, alternatively, the host cells can be in suspension.

Host cells as provided herein can comprise a constitutive secretory pathway, where proteins, e.g., proteins of interest expressed by the cell or by a library, are secreted from the interior of the cell either to be expressed on a cell or viral membrane surface or to be fully secreted as soluble polypeptides. In certain aspects, proteins of interest expressed on or in a biological membrane, e.g., an IMP, are expressed on the surface of an enveloped virus produced by the host cell, e.g., an extracellular enveloped vaccinia virus, or EEV. IMPS can follow the same pathway as fully secreted forms or proteins, passing through to the ER lumen, except that they can be retained in the ER membrane by the presence of one or more stop-transfer signals, or “transmembrane domains.” Transmembrane domains are hydrophobic stretches of about 20 amino acids that adopt an alpha-helical conformation as they transverse the membrane. Membrane embedded proteins are anchored in the phospholipid bilayer of the plasma membrane. Transmembrane forms of polypeptides of interest, e.g., membrane-anchored immunoglobulin heavy chain polypeptides typically utilize amino terminal signal peptides as do fully secreted forms.

Signal peptides, transmembrane domains, and cytosolic or “intra-membrane” domains are known for a wide variety of membrane bound and/or fully secreted proteins.

Suitable transmembrane domains can include but are not limited to the TM domain of the vaccinia virus EEV-specific HA protein A56R, or the EEV-specific vaccinia virus transmembrane proteins A33R, A34R, A36R, or B5R. See, e.g., U.S. Patent Appl. Publ. No. 2013/0288927, published Oct. 31, 2013, and incorporated herein by reference in its entirety. In certain aspects the EEV specific protein can be anchored to the inner surface of the viral envelope via a palmitoyl group, e.g., the vaccinia virus protein F13L. In some embodiments, transmembrane domains are referred to herein as “anchor proteins,” for example, “poxvirus anchor proteins.”

As used herein, the term “binding molecule” refers in its broadest sense to a molecule that specifically binds to a receptor, e.g., an epitope or an antigenic determinant. As described further herein, a binding molecule can comprise one or more “antigen binding domains” described herein. A non-limiting example of a binding molecule is an antibody or fragment thereof that retains antigen-specific binding.

The terms “binding domain” and “antigen binding domain” are used interchangeably herein and refer to a region of a binding molecule that is necessary and sufficient to specifically bind to an epitope. For example, an “Fv,” e.g., a variable heavy chain and variable light chain of an antibody, either as two separate polypeptide subunits or as a single chain, is considered to be a “binding domain.”

Other antigen binding domains include, without limitation, the variable heavy chain (VHH) of an antibody derived from a camelid species, or six immunoglobulin complementarity determining regions (CDRs) expressed in a fibronectin scaffold.

The terms “antibody” and “immunoglobulin” can be used interchangeably herein. An antibody (or a fragment, variant, or derivative thereof as disclosed herein) includes at least the variable region of a heavy chain (e.g., for camelid species) or at least the variable regions of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen binding fragment of an antibody to a full sized antibody, e.g., an IgG antibody that includes two complete heavy chains and two complete light chains.

The term “immunoglobulin” comprises various broad classes of polypeptides that can be distinguished biochemically. Those skilled in the art will appreciate that heavy chains are classified as gamma, mu, alpha, delta, or epsilon, ( [symbol] , μ, α, [symbol] , ε) with some subclasses among them (e.g., [symbol] 1- [symbol] 4 or [symbol] 1- [symbol] 4 or α1-α2)). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, IgA2, etc. are well characterized and are known to confer functional specialization.

Light chains are classified as either kappa or lambda (K, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure.

The term “epitope” includes any molecular determinant capable of specific binding to an antibody. In certain aspects, an epitope can include chemically active surface groupings of molecules such as amino acids, sugar side chains, phosphoryl, or sulfonyl, and, in certain aspects, can have three dimensional structural characteristics, and or specific charge characteristics. An epitope is a region of a target that is bound by an antibody.

The term “target” is used in the broadest sense to include substances that can be bound by a binding molecule. A target can be, e.g., a polypeptide, a nucleic acid, a carbohydrate, a lipid, or other molecule. Moreover, a “target” can, for example, be a cell, an organ, or an organism that comprises an epitope bound that can be bound by a binding molecule.

Both the light and heavy chains are divided into regions of structural and functional homology. The terms “constant” and “variable” are used functionally. In this regard, it will be appreciated that the variable regions (which can be called “variable domains” interchangeably herein) of both the variable light (VL) and variable heavy (VH) chain portions determine antigen recognition and specificity. As used herein, the term “antibody variable gene or fragment thereof” refers to the gene or portion thereof encoding a VL or VH or fragment thereof of an antibody. Conversely, the constant domains of the light chain (CL) and the heavy chain (e.g., CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 (or CH4 in the case of IgM) and CL domains are at the carboxy-terminus of the heavy and light chain, respectively.

The six “complementarity determining regions” or “CDRs” present in an antibody antigen binding domain are short, non-contiguous sequences of amino acids that are specifically positioned to form the antigen binding domain as the antibody assumes its three-dimensional configuration in an aqueous environment. The remainder of the amino acids in the antigen binding domain, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. Immunoglobulin variable domains can also be analyzed, e.g., using the IMGT information system (www://imgt.cines.fr/) (IMGT.RTM./V-Quest) to identify variable region segments, including CDRs. (See, e.g., Brochet et al., Nucl. Acids Res., 36:W503-508, 2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

Binding molecules, e.g., antibodies or antigen binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′).sub.2, Fd, Fvs, single-chain Fvs (scFv), single-chain antibodies, disulfide-linked Fvs (sdFv), single domain antibodies such as camelid VHH antibodies, fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule. Also contemplated are immunoglobulin new antigen receptor (IgNAR) isotypes that are bivalent and comprise a single chain that includes an IgNAR variable domain (VNAR). (See, Walsh et al., Virology 411:132-141, 2011).

By “specifically binds,” it is generally meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

As used herein, the term “affinity” refers to a measure of the strength of the binding of an individual epitope with one or more antigen binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen binding domains and an antigen. See, e.g., Harlow at pages 29-34. Avidity is related to both the affinity of individual antigen binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

As used herein, the term “heavy chain subunit” or “heavy chain domain” includes amino acid sequences derived from an immunoglobulin heavy chain, a binding molecule, e.g., an antibody comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof.

As used herein, the term “light chain subunit” or “light chain domain” includes amino acid sequences derived from an immunoglobulin light chain. The light chain subunit includes at least one of a VL or CL (e.g., CK or Cλ) domain.

Binding molecules, e.g., antibodies or antigen binding fragments, variants, or derivatives thereof can be described or specified in terms of the epitope(s) or portion(s) of an antigen that they recognize or specifically bind. The portion of a target antigen that specifically interacts with the antigen binding domain of an antibody is an “epitope,” or an “antigenic determinant.” A target antigen can comprise a single epitope or at least two epitopes, and can include any number of epitopes, depending on the size, conformation, and type of antigen.

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature). Although the reading frame is thus made continuous throughout the fused segments, the segments can be physically or spatially separated by, for example, in-frame linker sequence. For example, polynucleotides encoding an IMP and a vaccinia virus EEV-specific protein can be fused, in-frame, but be separated by a polynucleotide encoding a linker or spacer, as long as the “fused” open reading frames are co-translated as part of a continuous polypeptide.

As used herein, the term “hemagglutinin tag” or “HA tag” is a protein derived from a human influenza hemagglutinin surface glycoprotein (HA) corresponding to amino acids 98-106. The HA tag is extensively used as a general epitope tag in expression vectors. Recombinant proteins can be engineered to express the HA tag, which does not appear to interfere with the bioactivity or the biodistribution of the recombinant protein. This tag facilitates the detection, isolation, and purification of the protein of interest.

In the context of polypeptides, a “linear sequence” or a “sequence” is an order of amino acids in a polypeptide from the amino or N-terminus to the carboxyl or C-terminus, in which amino acids that neighbor each other in the sequence are contiguous in the primary structure of the polypeptide.

A portion of a polypeptide that is “amino-terminal” or “N-terminal” to another portion of a polypeptide is that portion that comes earlier in the sequential polypeptide chain. Similarly a portion of a polypeptide that is “carboxy-terminal” or “C-terminal” to another portion of a polypeptide is that portion that comes later in the sequential polypeptide chain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The term “eukaryote” or “eukaryotic organism” is intended to encompass all organisms in the animal, plant, and protist kingdoms, including protozoa, fungi, yeasts, green algae, single celled plants, multi celled plants, and all animals, both vertebrates and invertebrates. The term does not encompass bacteria or viruses. A “eukaryotic cell” is intended to encompass a singular “eukaryotic cell” as well as plural “eukaryotic cells,” and comprises cells derived from a eukaryote.

As used herein, the term “identify” refers to methods in which a desired molecule, e.g., an antibody or antibody-like molecule that binds to a target protein of interest, e.g., an integral membrane protein of interest, is differentiated from a plurality or library of such molecules. Identification methods include “selection” and “screening” or “panning.” As used herein, “selection” methods are those in which the desired molecules can be directly separated from the library, e.g., via drug resistance. As used herein, “screening” or “panning” methods are those in which pools comprising the desired molecules are subjected to an assay in which the desired molecule can be detected. Aliquots of the pools in which the molecule is detected are then divided into successively smaller pools which are likewise assayed, until a pool which is highly enriched for the desired molecule is achieved.

IMP fusion proteins as provided herein are produced in poxvirus vectors, e.g., vaccinia virus vectors. The term “poxvirus” includes any member of the family Poxviridae. See, for example, B. Moss in: Virology, 2d Edition, B. N. Fields, D. M. Knipe et al., Eds., Raven Press, p. 2080 (1990). The genus of orthopoxvirus includes, e.g., vaccinia virus, variola virus (the virus that causes smallpox), and raccoon poxvirus. Vaccinia virus is the prototype orthopoxvirus and has been developed and is well-characterized as a vector for the expression of heterologous proteins. The IMP fusion proteins can be produced as disclosed in U.S. Pat. No. 10,550,199, incorporated herein in its entirety.

In those embodiments where poxvirus vectors, in particular vaccinia virus vectors, are used to express IMP fusion proteins as provided herein, any suitable poxvirus vector can be used, e.g., vaccinia virus, fowl pox, or rabbit pox vectors. Polynucleotides encoding IMP fusion proteins as provided herein can be inserted into poxvirus vectors, particularly vaccinia virus vectors or fowl pox virus vectors, under operable association with a transcriptional control region which functions in the cytoplasm of a poxvirus-infected cell. Suitable poxvirus vectors include wild-type vaccinia virus, e.g., strain Western Reserve or WR, or attenuated vaccinia virus, e.g., modified vaccinia Ankara (MVA) (Mayr, A. et al., Infection 3:6-14 (1975)).

At least six virus-encoded proteins have been reported as components of the EEV envelope membrane. Of these, four proteins (A33R, A34R, A56R, and B5R) are glycoproteins, one (A36R) is a nonglycosylated transmembrane protein, and one (F13L) is a palmitoylated peripheral membrane protein. See, e.g., Lorenzo et al., Journal of Virology 74 (22):10535 (2000). During infection, these proteins localize to the Golgi complex, where they are incorporated into infectious virus that is then transported and released into the extracellular medium. As provided herein, IMP fusion proteins are directed to and expressed on the EEV membrane as a fusion protein with an EEV-specific protein, e.g., F13L or A56R.

The A56R protein is the vaccinia virus hemagglutinin and is a standard type I integral membrane protein comprising an amino-terminal extracellular (“extra-membrane”) domain, a single transmembran00e domain, and a cytoplasmic (“intra-membrane”) domain. A56R comprises an N-terminal signal peptide of about 33 amino acids, an Ig-like domain extending from about amino acid 34 to about amino acid 103, a stalk region extending from about amino acid 121 to about amino acid 275, a transmembrane domain extending from about amino acid 276 to about amino acid 303, and an cytoplasmic (“inter-membrane”) domain extending from about amino acid 304 to amino acid 314. See DeHaven et al., J. Gen Virol. 92:1971-1980 (2011). A56R is presented as SEQ ID NO: 1.

A56R:
(SEQ ID NO: 1)
MTRLPILLLLISLVYATPFPQTSKKIGDDATLSCNRNNTNDYVVMSAWYK
EPNSIILLAAKSDVLYFDNYTKDKISYDSPYDDLVTTITIKSLTARDAGT
YVCAFFMTSTTNDTDKVDYEEYSTELIVNTDSESTIDIILSGSTHSPETS
SKKPDYIDNSNCSSVFEIATPEPITDNVEDHTDTVTYTSDSINTVSASSG
ESTTDETPEPITDKEDHTVTDTVSYTTVSTSSGIVTTKSTTDDADLYDTY
NDNDTVPPTTVGGSTTSISNYKTKDFVEIFGITALIILSAVAIFCITYYI
YNKRSRKYKTENKV

As used herein, a “solid support” is any support capable of binding an EEV, which can be in any of various forms, as is known in the art. Well-known supports include tissue culture plastic, glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of this disclosure. The support material can have virtually any structural configuration as long as the coupled EEV is capable of binding to a displayed binding molecule such as an antibody. Thus, the support configuration can be spherical, as in a bead, or cylindrical, as in the inside surface of a test tube, or the external surface of a rod. Alternatively, the surface can be flat such as a sheet, test strip, etc. Typical supports include beads, e.g., magnetic polystyrene beads such as DYNABEADS® that can be pulled out of suspension by a magnet. The support configuration can include a tube, bead, microbead, well, plate, tissue culture plate, petri plate, microplate, microtiter plate, flask, stick, strip, vial, paddle, etc., etc. A solid support can be magnetic or non-magnetic. Those skilled in the art will know many other suitable carriers for binding EEV as provided herein, or will be able to readily ascertain the same. In certain aspects, EEV as provided herein can be attached to the solid support via reaction with, e.g., tosyl groups, epoxy groups, carboxylic acid groups, or amino groups attached to the surface. For example, EEV can be attached to the surface of tosyl-activated magnetic beads, e.g., MYONE™ tosylactivated beads. Alternatively, the EEV can be biotinylated and attached to a streptavidin solid surface, e.g., streptavidin coated magnetic beads. Any solid support can be used in the methods of the disclosure.

The poxvirus antigen constructs of the disclosure can be used in phage panning, for example, or cell sorting applications to provide complex antigens such as GPCRs and ion channels in native conformation for antibody discovery. This disclosure provides methods to select binding molecules, e.g., antibodies, antigen-binding antibody fragments, or antibody-like binding molecules that bind to the complex antigens of the disclosure, e.g., multi-pass membrane proteins of interest.

For certain aspects of the disclosure, a display library is used in the methods to select binding molecules. Any display library comprising a plurality of binding domains, e.g., antibodies, antibody like molecules or other binding molecules is suitable for use in the methods of the disclosure. For example, the display library can be a phage display library, a yeast display library or a library constructed in a vaccinia virus vector as described elsewhere herein.

For certain embodiments of the disclosure, an animal, such as a chicken, shark, mammal, etc. e.g., a mouse, is immunized one or more times with a target antigen to raise antibodies specific to the target antigen, e.g., a target IMP. In some embodiments, the target antigen comprises an antigen virion comprising a poxvirus that expresses an integral membrane protein (IMP)—extracellular enveloped virion (EEV) fusion protein in native conformation as part of the outer envelope membrane of the EEV. In some embodiments B cells are isolated from the immunized animal, e.g., chicken, shark, mammal, and sorted to isolate antigen-specific B cells that express IgG that specifically binds target IMP. Variable heavy chain genes and/or variable light chain genes can be isolated and cloned from individual sorted B cells. Platforms used to discover and isolate target-specific antibodies from single B cells include, without limitation, hybridoma techniques, memory B cell and adipose stem cell (ASC) culture techniques, membrane-bound B cell receptor (BCR) staining of B cells with antigen, single B cell screening methodologies, B cell replica methodologies, single B cell repertoire analysis and clonal expansion-guided identification, for example. (Pedrioli et al., Trends in Immunology, Single B cell technologies for monoclonal antibody discovery, 2021, vol. 42, no. 12; incorporated herein in its entirety).

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described can be followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W. H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevier, Amsterdam); Goldsby et al., eds. (2000) Kuby Immunology (4th ed.; H. Freeman & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall, 2003); Harlow and Lane (1988) Antibodies: A Laboratory Manual (Cold Spring Harbor Press); Dieffenbach and Dveksler (2003) PCR Primer (Cold Spring Harbor Press).

All of the references cited above, as well as all references cited herein, are incorporated herein by reference in their entireties. The following examples are offered by way of illustration and not by way of limitation.

### EXAMPLES

### Example 1: Preparation of EEV Coupled Polystyrene Streptavidin Fluorescent Beads

Extracellular enveloped virion (EEV) expressing antigen of interest (e.g., CD20, CD39, SEMA4D) was prepared as described in U.S. Pat. No. 10,577,427, incorporated herein in its entirety. To couple Fowl Pox Virus (FPV) EEV to polystyrene beads, the virus was captured with a purified and biotinylated chicken anti-FPV antibody (LSBio). For the modified vaccinia virus Ankara (MVA) EEV, the virus was directly biotinylated using FSL Biotin (Sigma) and next coupled to the streptavidin coated beads. Fluorescent polystyrene beads coated with streptavidin (Spherotech Cat. No. SVFP-1068-5 and SVFP-0552-5) were vortexed and sonicated and an appropriate volume of beads was removed and added to 1 mL of 1% BSA in 1×PBS, pH 7.2. The beads were pelleted by centrifugation at 13,000×g for 20 minutes and the supernatant removed. The beads were resuspended in 200 ul of 1% BSA in 1×PBS, pH 7.2 and 25 ug of biotinylated capture antibody was added per 1e8 beads. The beads were then rotated at Room Temperature for 2 hours. The beads were washed twice with 1% BSA, 1X PBS pH 7.2 with centrifugation at 13,000×g used to pellet the beads. The beads were resuspended in 400 uL of 1% BSA in 1×PBS, pH 7.2 and antigen EEV was added to the beads at a beads-to-virus ratio 5:1 or 1:1. The beads+EEV were rotated overnight at Room Temperature in the dark. The beads were washed once with 1% BSA in 1×PBS, pH 7.2 and then blocked with 1 ml of 1% BSA, 2% FBS in 1×PBS, pH 7.2. Beads were rotated overnight at Room Temperature in the dark to completely block. The beads were then pelleted by centrifugation at 13,000×g 20 min and resuspended in 1% BSA in 1X PBS, pH7.2 at their original starting volume. Beads+EEV were stored at 4° C. in foil until use.

### Example 2: V Genes for CD20, CD39 and Sema4D (CD100) Cloned into an A56R Vector

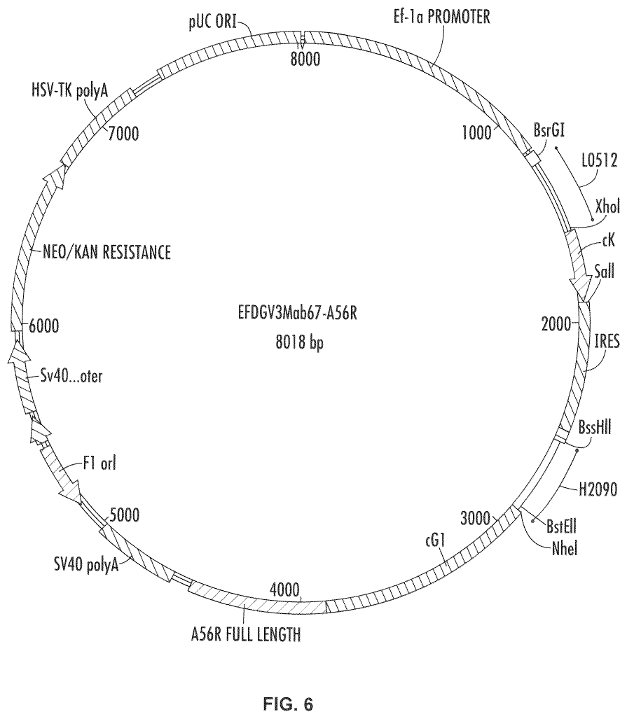

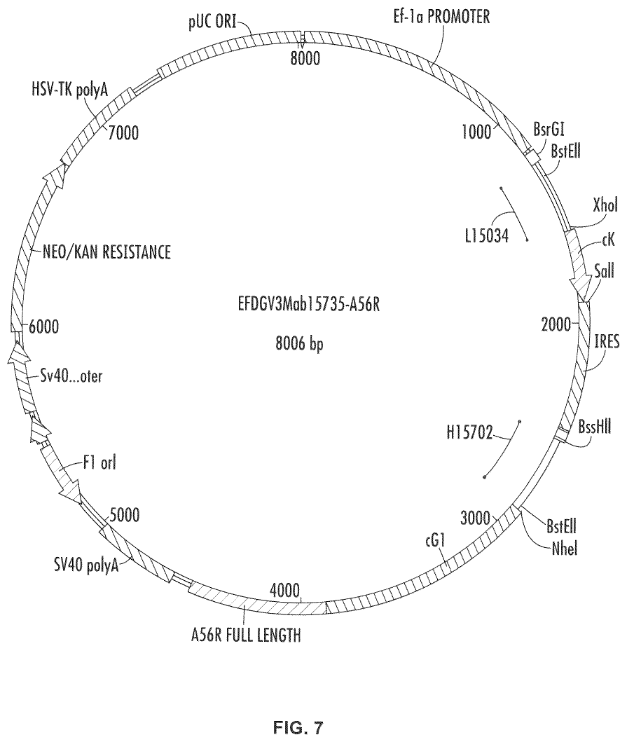

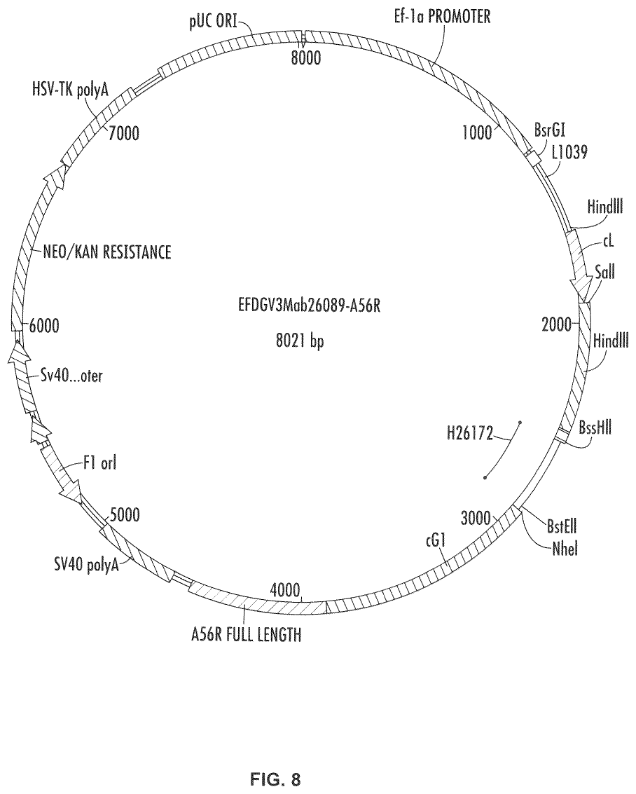

V-genes from sorted B cells were cloned and sequenced for display library generation. Vectors were generated as shown in FIGS. 6, 7, and 8. The amino acid sequence of each of the vectors shown in the figures is shown below.

### EFDGV3-Mab67-A56R (CD100)

(SEQ ID NO: 2)
MGWSCIILFLVATATGVHSDIVMTQSPASLAVSLGQRATISCKASQSVDY
DGDSYMNWYQQKPGQPPKLLIYAASNLESGIPARFSGSGSGTDFTLNIHP
VEEEDAATYYCQQSNEDPYTFGGGTKLEIKRTVAAPSVFIFPPSDEQLKS
GTASVVCLLNNFYPREAKVQWKVDNALOSGNSQESVTEQDSKDSTYSLSS
TLTLSKADYEKHKVYACEVTHQGLSSPVTKSFNRGEC

### Underline=Signal Sequence

Bold=Mab 67 Light Variable sequence, Italics=Human Kappa constant region

-IRES Element-
(SEQ ID NO: 3)
MGWSCIILFLVATATGAHSQVQLQQSGPELVKPGASVKISCKASGYSFSD
YYMHWVKQSPENSLEWIGQINPTTGGASYNQKFKGKATLTVDKSSSTAYM
QLKSLTSEESAVYYCTRYYYGRHFDVWGQGTTVTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGKTSTTNDTDKVDYEEYSTELIVNTDSESTIDIIL
SGSTHSPETSSKKPDYIDNSNCSSVFEIATPEPITDNVEDHTDTVTYTSD
SINTVSASSGESTTDETPEPITDKEDHTVTDTVSYTTVSTSSGIVTTKST
TDDADLYDTYNDNDTVPPTTVGGSTTSISNYKTKDFVEIFGITALIILSA
VAIFCITYYIYNKRSRKYKTENKV

### Underline=Signal Sequence

Bold=Mab 67 heavy Variable sequence, italics=Human Gamma constant region

Bold and Italics=A56R

EFDGV3-Mab15735-A56R (CD20)

(SEQ ID NO: 4)
MGWSCIILFLVATATGVHSQIVLTQSPAIMSASPGEKVTMTCSASSSVYY
MHWYQQKSGTSPKRWIYDTSKLASGVPARFSGSGSGTSYSLTISSMEAED
AATYYCQQWSSNPLTFGGGTKLEIKRTVAAPSVFIFPPSDEQLKSGTASV
VCLLNNFYPREAKVQWKVDNALQSGNSQESVTEQDSKDSTYSLSSTLTLS
KADYEKHKVYACEVTHQGLSSPVTKSFNRGEC

Underline=Signal Sequence

Bold=Mab 15735 light variable sequence, Italics=Human Kappa constant region

-IRES Element-
(SEQ ID NO: 5)
MGWSCIILFLVATATGAHSQVQLQQSGAELVRPGASVKMSCKASGYTFTS
YNMHWVKQTPRQGLEWIGAIYPGNGDTSYNQKFKGKATLTVDKSSSTAYM
QLSSLTSEDSAVYFCARSGTGSYAMDYWGQGTTVTVSSASTKGPSVFPLA
PSSKSTSGGTAALGCLVKDYFPEPVTVSWNSGALTSGVHTFPAVLOSSGL
YSLSSVVTVPSSSLGTOTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHODWLNGKEYKCKVSNKALP
APIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTOKSLSLSPGKTSTTNDTDKVDYEEYSTELIVNTDSESTIDII
LSGSTHSPETSSKKPDYIDNSNCSSVFEIATPEPITDNVEDHTDTVTYTS
DSINTVSASSGESTTDETPEPITDKEDHTVTDTVSYTTVSTSSGIVTTKS
TTDDADLYDTYNDNDTVPPTTVGGSTTSISNYKTKDFVEIFGITALIILS
AVAIFCITYYIYNKRSRKYKTENKV

Underline=Signal Sequence

Bold=Mab 15735 heavy Variable sequence, italics=Human Gamma constant region

Bold and italics=A56R

EFDGV3-Mab26089-A56R (CD39)

(SEQ ID NO: 6)
MGWSCIILFLVATATGVHSSYELTQPPSVSVSPGQTASITCSGDKLGDKY
ASWYQQKPGQSPVLVIYQDSKRPSGIPERFSGSNSGNTATLTISGTQAMD
EADYYCQAWDSSTAWVFGGGTKLTVLGQPKAAPSVTLFPPSSEELQANKA
TLVCLISDFYPGAVTVAWKADSSPVKAGVETTTPSKQSNNKYAASSYLSL
TPEQWKSHRSYSCQVTHEGSTVEKTVAPTECS

Underline=Signal Sequence

Bold=Mab 26089 light variable sequence, Italics=Human lambda constant region

-IRES Element-
(SEQ ID NO: 7)
MGWSCIILFLVATATGAHSQVQLVQSGAEVKKPGASVKVSCKASGYTFTS
YYMHWVRQAPGQGLEWMGIINPSGGSTSYAQKFQGRVTMTRDTSTSTVYM
ELSSLRSEDTAVYYCARDRYSRYYDYLGFDYWGQGTLVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPGKTSTTNDTDKVDYEEYSTELIVNTDSEST
IDIILSGSTHSPETSSKKPDYIDNSNCSSVFEIATPEPITDNVEDHTDTV
TYTSDSINTVSASSGESTTDETPEPITDKEDHTVTDTVSYTTVSTSSGIV
TTKSTTDDADLYDTYNDNDTVPPTTVGGSTTSISNYKTKDFVEIFGITAL
IILSAVAIFCITYYIYNKRSRKYKTENKV

Underline=Signal SequencE

Bold=Mab26089 heavy variable sequence, italics=Human Gamma constant

Bold and italics=A56R

Generation of Stable CHO Cell Line Expressing Membrane Bound Antibody

One day prior to transfection, CHO cells were seeded into 6 well plates (0.8E6/well). Cells were transfected with 4 ug of vector DNA using Lipofectamine 2000 reagent following manufacturer's instructions. Cells with No Vector were included as a control. The following day, the cells were harvested and dispensed into T175 flasks with DMEM medium containing 10% FBS, 1 mM HEPES and G418 for drug selection (0.6 mg/ml). Media containing drug was changed every 2-3 days to maintain selection pressure. When the No Vector cells had died off, the transfectants were stained using anti-Fab antibody for Fluorescence Activated Cell Sorting (FACS) on the BD FACS Aria sorter. Cells with high antigen expression were collected, cultured and post-sort enrichment was determined by flow cytometry.

Staining with Fluorescent Yellow/Blue Beads

Cells were collected using Accutase dissociation reagent, counted, pelleted at 300×g for 5 minutes and resuspended in blocking buffer (1% BSA, 2% FBS in 1×PBS, pH 7.2) at 1 million cells/ml. Cells were kept on ice for 1 h. The cells were next pelleted and resuspended in 1 ml of 1% BSA, 100 Units Heparin, 1X PBS, pH7.2 per 1 million cells and incubated for 20 minutes at Room Temperature. Cells were then dispensed to flow staining tubes (0.2 ml each) and EEV coupled fluorescent beads were added to each tube. Cells were incubated with beads for 1 hour at room temperature in the dark with occasional mixing. The cells were next washed twice with 1×PBS, pH 7.2, 1% BSA and resuspended in 1×PBS, pH 7.2, 1% BSA with anti-Fab-FITC or anti-Fab-APC. Cells were incubated at 4° C. for 30 minutes and then washed twice with 1×PBS, pH 7.2, 1% BSA and fixed with 0.5% paraformaldehyde in 1×PBS, pH 7.2, 1% BSA before running on the BD FACS Canto II with propidium iodide for live/dead discrimination.

### Example 3: Flow Staining and Sorting of CHO Cells Stably Expressing Membrane Bound IgG Library

A membrane bound Fab-A56 plasmid library was generated from outputs from phage panning on antigen EEV.

Preparing Membrane Bound Mini Library (MBML) from Phage Pans:

Bacterial pellet containing phagemid was obtained from pan and plasmid DNA was extracted (Qiagen HiSpeed Maxiprep kit, cat #12662). Expression cassette containing the linked heavy and light chains (variable light/constant light-RBS element-Variable Heavy) was subcloned as a pool into mammalian expression dual gene vector pEFDGV3-Mab-A56R(Kan) using BsrG1 and NheI restriction sites and standard ligation and transformation protocols (pEFDGV3 contains the gamma I CH1 constant domain to complete the antibody cassette upon cloning) The library was plated on standard 150 mm LB AGAR plates containing 50 mg/mL Kanamycin (LB-Kan50) and incubated overnight at 37° C. Control vector only plate was included. Colonies were counted and background determined. At least 5000 colonies were harvested from the plates (10 ML LB/Glycerol per plate was applied to each plate and colonies were gently lifted from the agar surface using a sterile cell scraper) and plasmid DNA was extracted using Qiagen plasmid DNA kit. This pool was subsequently digested with SalI/BssHI to remove the RBS element and replace it with an IRES element for mammalian co-expression. Transformations were plated on 150 mm LB-Kan50 plates and incubated overnight at 37° C. At least 5000 colonies were harvested in bulk and plasmid DNA isolated. DNA concentration was measured by nanodrop and the DNA was handed off for transfection.

Approximately 10 million CHO cells were transfected in bulk to cover the diversity of the plasmid library using Lipofectamine 2000 and incubated 24 hours. Cells were then collected and dispensed into three T300 flasks with DMEM medium containing 10% FBS, 1 mM HEPES and G418 (0.6 mg/ml). Media containing drug was changed every 2-3 days to maintain selection pressure. After 2-3 weeks in drug selection culture, cells were collected using Accutase dissociation reagent and counted. A minimum of 6 million cells were used for sorting. Cells were pelleted at 300 g for 5 minutes. The media was aspirated and then the cells were resuspended in blocking buffer (1% BSA, 2% FBS in 1×PBS, pH 7.2) on ice for 1 hour. The cells were pelleted and resuspended in 1 ml of 1% BSA, 100 U Heparin, 1X PBS, pH7.2 per 2.5 million cells and incubated for 20 minutes at Room Temperature. Cells were then dispensed to flow staining tubes (0.4 ml each) and EEV coupled fluorescent yellow beads were added to each tube (40 μl/tube). The cells+beads were incubated at Room Temperature for 45 min-1 hour in the dark with occasional mixing. The cells were washed twice with 1X PBS, pH 7.2, 1% BSA, and then resuspended in 0.4 ml of the same with anti-Fab-APC. Cells were incubated for 30 minutes at 4° C. and then washed twice with 1X PBS, pH 7.2, 1% BSA., resuspended in 1 ml of the same and filtered through a 40 μm filter mesh before being sorted. Controls were used to create gates to capture antigen EEV+/anti-Fab+ cells. Sorted cells were pelleted and washed with 1×PBS. Cell pellets were stored at −minus 20° C. before genomic DNA extraction (Qiagen QiaAmp BloodDNA Mini) and the mab expression cassette was amplified using PCR (Takara Advantage cDNA polymerase2) to include restriction sites for cloning.

Libraries with human Constant regions, mab expression cassette were amplified with the following primers

(SEQ ID NO: 8)
VLsigFWD-GATGGAGCTGTATCATCCTCTTCTTGGTAG
(SEQ ID NO: 9)
hVHrev-TGGTGTTGCTGGGCTTGTGATTCA

Libraries with fully mouse expression, mab expression cassette were amplified with the following primer pairs.

(SEQ ID NO: 10)
mVLfwd-AGTTAGGCCAGCTTGGCACTTGATG
(SEQ ID NO: 11)
mGlrev-ACTGTCACTGGCTCAGGGAAATAGC
(SEQ ID NO: 10)
mVLfwd-AGTTAGGCCAGCTTGGCACTTGATG
(SEQ ID NO: 11)
mG2a-rev-AGGAGCCAGTTGTATCTCCACACA

Cycle titration was performed to determine appropriate amplification conditions to minimize bias. PCR product (2000 bp) was digested with either BsrG1/Nhe (human V-genes) or BsiW1/Nhe1 for fully mouse and the cassette subcloned into our mammalian double gene expression vector EVDGV3 (human), EFDGVmVKG1 or EFDGVmVKG2a (mouse).

Transformations were plated on 100 mm LB-Kan50 plates at various densities to ensure good colony separation and incubated overnight at 37 degrees C. 94 colonies were picked into 96 well deep well growth plate containing 1.6 mL/well LB/Kan50 and grown for 22 hrs at 37° C. A spot plate was arrayed to allow for future propagation of each individual clone in the future. Plasmid DNA was isolated in this format using the Qiagen turbo 96 kit. DNA concentration was measured by nanodrop and averaged to assign a single plate concentration and the DNA was handed off for transfection.

DNA was sequenced at Genewiz using two primers—Ef1F forward primer (5′-TGGAATTTGCCCTTTTTGAG-3′ (SEQ ID NO: 13)) for the light chain variable region and cGS reverse primer (5′ AAGTAGTCCTTGACCAGGCAGCC-3′ (SEQ ID NO: 14)) for the human heavy chain variable region

ECMVIRESFWD primer (SEQ ID NO: 15) (5′-TACATGTGTTTAGTCGAG-3′ (SEQ ID NO: 16)) for the mouse heavy chain variable region and into the constant.

### Example 4: Flow Staining of Immunized Mouse B Cells Using Antigen EEV

Balb/c mice were immunized three or four times with EEV MVA-T7-CD20-G-F. Six days after last immunization, mice were sacrificed and spleens were harvested for B cell isolation following the Miltenyi Biotec B cell CD19+ positive isolation protocol. B cells were blocked with 1% BSA, 2% FBS in 1×PBS, pH 7.2 for one hour on ice. The B cells were pelleted and resuspended in 0.5 ml of 1% BSA in 1×PBS, pH 7.2. Fluorescent Blue beads (25u1) coupled with FPV-H5-CD20-F EEV were added to the B cells and incubated 45 min-1 hour at Room Temperature in the dark with gentle mixing every 15 minutes. The B cells were washed twice with 2 mL of 1% BSA in 1X PBS, pH7.2 and resuspended in 0.5 ml of the same. Secondary antibodies were added: anti-B220-FITC, anti-mIgG1-BV421, anti-mIgG2a and 2b-BV421 and anti-mIgM-PerCP-eFluor 710 and incubated for 30 minutes at 4° C. B cells were washed twice with 2 mL 1% BSA in 1×PBS, pH 7.2 and resuspended in the same before passing through a 40 μm filter mesh and sort. Gating was set up to collect the B220+/IgM−/IgG+/CD20 antigen specific B cells. Sorted cells were pelleted, washed with PBS and stored at 4° C. before cDNA processing.

Phage Library Construction from Sorted B Cells

1700 Sorted B cells (for Y189) were stored in RNAlater™ (ThermoFisher cat #AM7020). RNA was extracted using RNAeasy micro kit (Qiagen74004), DNAse-treated and quantified by nanodrop. cDNA was prepared using standard protocols followed by RNAase treatment. For cDNA synthesis, the cDNA was primed using primers specific to the constant domain of mouse gamma constant 1 and constant IgG2a gene and mouse kappa constant. Heavy chain variable regions were PCR amplified using standard methods and utilizing a mix of mouse VH gene and JH gene primer containing BssHII and BsteII restriction sites. The PCR product was gel purified. Light chain variable regions were PCR amplified using standard methods and utilizing a mix of mouse VK gene and JK gene primers containing ApaL1 and Xho1 restriction sites. The Heavy chain PCR product was gel purified, V-genes were bulk cloned into a phagemid pool (pAD14huGLlights) at the BssHII/BsteII sites (pAD phagemid backbone in the pool containing 14 human germline variable light chains fused to human constant regions separated by a Ribosome Binding site (RBS)) using NxGen T4 DNA Ligase, Lucigen 3024-1. Ligation reactions were transformed via electroporation into TG1 Electrocompetent cells, Lucigen #60502-2, with 1 hr outgrowth and expanded culture at 37° C. for 5 hours with shaking in 2YXT buffer with glucose and ampicillin. Phagemid library was harvested by centrifugation at 4° C., 6200 rpm for 15 minutes. Pellets were re-suspended in freezing media (containing 2XYT, glycerol, glucose and Amp). Bacteria were plated to titer the library and a subset of phagemid were mini-prepped and sequenced for library quality control. A glycerol was expanded and a maxiprep done by standard procedures using Qiagen HiSpeed Plasmid Maxi kit-cat #12662. This DNA was used as the source of heavy chain V-genes for the final library.

The light chain PCR product was gel purified. V-genes were bulk cloned into a phagemid pool (pApAD) at the ApaL1/Xho1 sites (pApAD phagemid backbone in the pool containing human light constant fused to human constant regions with stuffer sequence for cloning variable regions separated by a Ribosome Binding site (RBS)) using NxGen T4 DNA Ligase, Lucigen 3024-1. Ligation reactions were transformed via electroporation into TG1 Electrocompetent cells, Lucigen #60502-2, with 1 hr outgrowth and expanded culture at 37° C. for 5 hours with shaking in 2YXT buffer with glucose and ampicillin. Phagemid library was harvested by centrifugation at 4° C., 6200 rpm for 15 minutes. Pellets were re-suspended in freezing media (containing 2XYT, glycerol, glucose and Amp). Bacteria were plated to titer the library and a subset of phagemid were mini-prepped and sequenced for library quality control. A glycerol was expanded and a maxiprep done by standard procedures. This DNA was used as the vector backbone for the final library. Maxiprep DNA from heavy and light libraries were digested with BssHII and Nhe1. The heavy chains were cloned into the stuffer region of the light library. NxGen T4 DNA Ligase, Lucigen 3024-1. Ligation reactions were transformed via electroporation into TG1 Electrocompetent cells, Lucigen #60502-2, with 1 hr outgrowth and expanded culture at 37° C. for 5 hours with shaking in 2YXT buffer with glucose and ampicillin. Phagemid library was harvested by centrifugation at 4° C., 6200 rpm for 15 minutes. Pellets were re-suspended in freezing media (containing 2XYT, glycerol, glucose and Amp). Bacteria were plated to titer the library (390 million clones) and a subset of phagemid were mini-prepped and sequenced for library quality control.

Primers Used for cDNA-Heavy Chains

mCGlrev
(SEQ ID NO: 17)
GGAGTTAGTTTGGGCAGCAGATCCA
mCG2arev
(SEQ ID NO: 18)
TCCAGAGTTCCAGGTCAAGGT

Primers Used for PCR Amplification Heavy Chains

mVH 1
(SEQ ID NO: 19)
AATATGCGCGCACTCCCAGGTCCAGCTGCAGCAGTct
mVH 2
(SEQ ID NO: 20)
AATATGCGCGCACTCCCAGGTGCAGCTGAAGGAGTC
mVH 3
(SEQ ID NO: 21)
AATATGCGCGCACTCCGAGGTGCAGCTTCAGGAG
mVH 4
(SEQ ID NO: 22)
AATATGCGCGCACTCCGAGGTGAAGCTTCTCGAGTCTG
mVH 5
(SEQ ID NO: 23)
AATATGCGCGCACTCCGAAGTGAAGCTGGTGGAGTCTG
mJH
(SEQ ID NO: 24)
GAGACGGTGACCGTGGTCCCTTGGCCCCA

Primers Used for cDNA-Light Chains

mcKrev2
(SEQ ID NO: 25)
TGCTCACTGGATGGTGGGAAGATGGA

Primers Used for PCR Amplification Light Chains

mJK1
(SEQ ID NO: 26)
CCGTTTGATCTCGAGCTTGGTGCCTCCACCGAA
mJK2
(SEQ ID NO: 27)
CCGTTTTATCTCGAGCTTGGTCCCCCCTCCGAA
mJK3
(SEQ ID NO: 28)
CCGTTTTATCTCGAGTCTGGTCCCATCACTGAA
mJK4
(SEQ ID NO: 29)
CCGTTTTATCTCGAGCTTTGTCCCCGAGCCGAA
mJK5
(SEQ ID NO: 30)
CCGTTTCAGCTCGAGCTTGGTCCCAGCACCGAA
mVKa_ApaLI
(SEQ ID NO: 31)
gctataGTGCACTCCGACATTGTGATGACCCAGTCTC
mVKb_ApaLI
(SEQ ID NO: 32)
gctataGTGCACTCCGACATTGTGATGACACAGTC
mVKc_ApaLI
(SEQ ID NO: 33)
gctataGTGCACTCCGATGTTGTGATGACCCA
mVKd_ApaLI
(SEQ ID NO: 34)
gctataGTGCACTCCGACATTGTGCTGACTCAGT
mVKe_ApaLI
(SEQ ID NO: 35)
gctataGTGCACTCCGACATTGTGCTGACCCAATCTC
mVKf_ApaLI
(SEQ ID NO: 36)
gctataGTGCACTCCCAAATTGTTCTCACCCAGTCTC
mVKg_ApaLI
(SEQ ID NO: 37)
gctataGTGCACTCCGACATCCAGATGACCCAGTCT

Phage library was made from a bacterial glycerol stock by a 1:100fold dilution into 2XYT media containing 2% glucose and 100 ug/mL Ampicillin at a volume necessary to achieve a 3fold coverage of library diversity. Library was grown shaking at 37° C. until an OD600 of approximately 0.5 was reached, at which point hyperphage was added at an MOI of 20. Following an hour of growth, bacteria were pelleted by centrifugation and resuspended in 2XYT media containing 50 ug/mL Kanamycin and 100 ug/mL Ampicillin and allowed to grow overnight for expansion of phage. Following centrifugation of the overnight cultures, enriched phage was precipitated from supernatant, concentrated by centrifugation, and resuspend in PBS for subsequent use in panning.

Phage display selection was done following standard methods (Toxins, 2018, Basics of Antibody Phage Display Technology, 10, 236) with some modifications for panning on virus. To select antibodies through phage panning, EEV was coupled to MyOne Tosylactivated Dynabeads (Invitrogen, 6550) by combining 3E8pfu EEV with 50 uL of beads in a final volume of 1 mL PBS, and rotated at 37° C. overnight. Unbound EEV was removed by pulling beads towards a magnet and washing. Beads were then blocked for 2 hours at 37° C. with PBS+10% FB S/1% BSA. For the initial round of selection, a phage Fab display library containing approximately 1E8 unique clones was added to coupled EEV for 2 hours at room temperature. Following extensive washing, phage bound to beads was added directly to TG1 cells for overnight expansion. Following centrifugation of the overnight TG1 cultures, enriched phage was precipitated from supernatant, concentrated by centrifugation, and resuspend in PBS for subsequent rounds of panning. For each additional round of panning, amount of input phage was decreased by a factor of 10 as well as depleted for multiple rounds on a control EEV, in order to remove anti-virus binding antibodies. Coupling EEV for depletion was done in an identical manner as for selection. Following rounds of enrichment, the V genes from the phage pool were subcloned into a mammalian expression vector while maintaining the VH and VL pairing that was in each phage as a minilibrary (ML).

Preparing Minilibrary (ML) from Phage Pans:

Bacterial pellet containing phagemid was obtained from phage pan and plasmid DNA is extracted (Qiagen HiSpeed Maxiprep kit, cat #12662). Expression cassette containing the linked heavy and light chains (variable light/constant light-RBS element-Variable Heavy) was sub cloned as a pool to mammalian expression dual gene vector pEFDGV3ApaL1 (Kan) using ApaL1 and NheI restriction sites and standard ligation and transformation protocols (pEFDGV3ApaL1 contains the heavy constant to complete the antibody cassette upon cloning) The library was plated on 4 standard 150 mm LB AGAR plates containing 50 mg/mL Kanamycin (LB-Kan50) and incubated overnight at 37° C. Control vector only plate is included. Colonies were counted and background determined. Approximately 5000 colonies were harvested from the plates (10 ML LB/Glycerol per plate is applied to each plate and colonies are gently lifted from the agar surface using a sterile cell scraper) and plasmid DNA was extracted using Qiagen plasmid DNA kit. This pool was subsequently digested with Sa1I/BssHI to remove the RBS element and replace it with an IRES element for mammalian co-expression. Transformations were plated on 100 mm LB-Kan50 plates at various densities to ensure good colony separation and incubated overnight at 37° C. 94 colonies were picked into 96 well deep well growth plate containing 1.6 mL/well LB/Kan50 and grown for 22 hrs at 37° C. A spot plate was arrayed to allow for future propagation of each individual clone in the future. Plasmid DNA was isolated in this format using the Qiagen turbo 96 kit. DNA concentration was measured by nanodrop and averaged to assign a single plate concentration and the DNA was handed off for transfection and testing of antibody by flow cytometry.

DNA was sequenced at Genewiz using two primers—Ef1F forward primer (5′-TGGAATTTGCCCTTTTTGAG-3′ (SEQ ID NO: 13)) for the light chain variable region and cGS reverse primer (5′ AAGTAGTCCTTGACCAGGCAGCC-3′ (SEQ ID NO: 14)) for the heavy chain variable region.

### Example 5: Flow Staining of Immunized Mouse B Cells

### CD20 and CD39

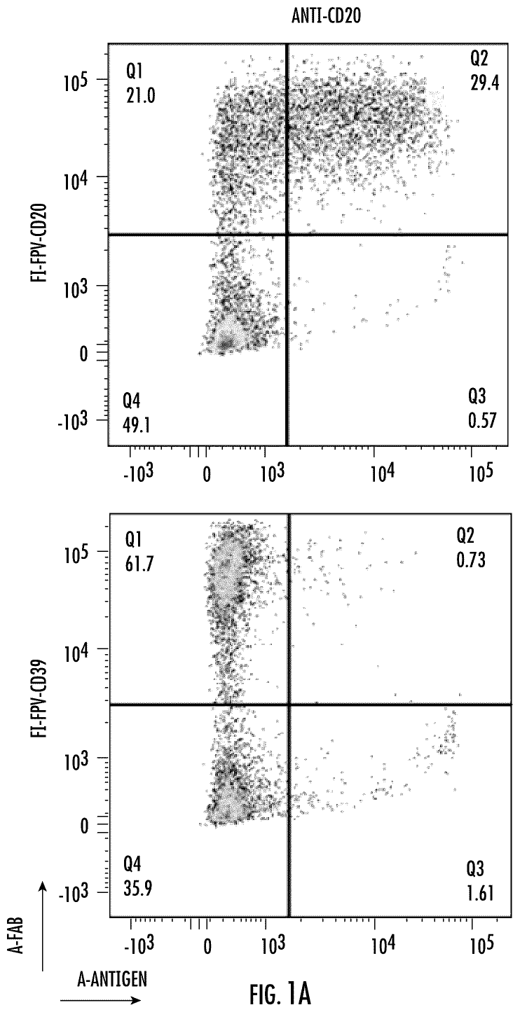

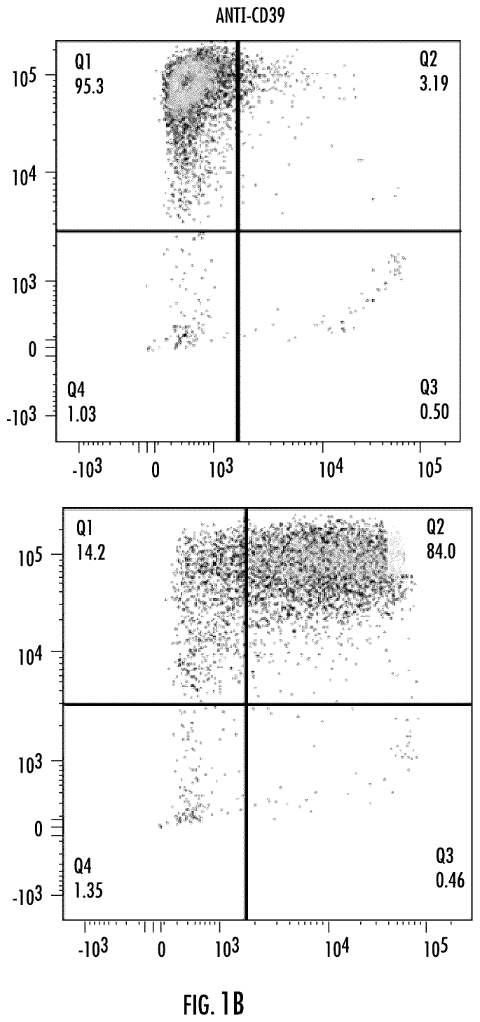

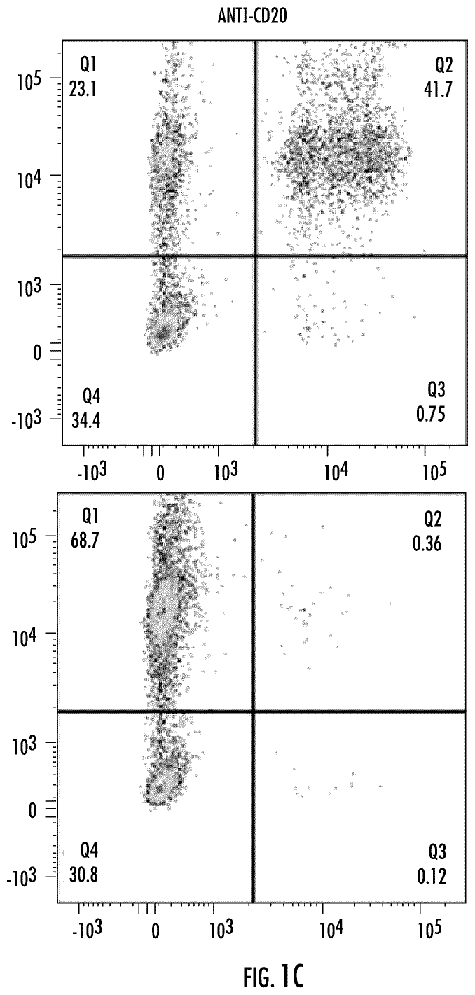

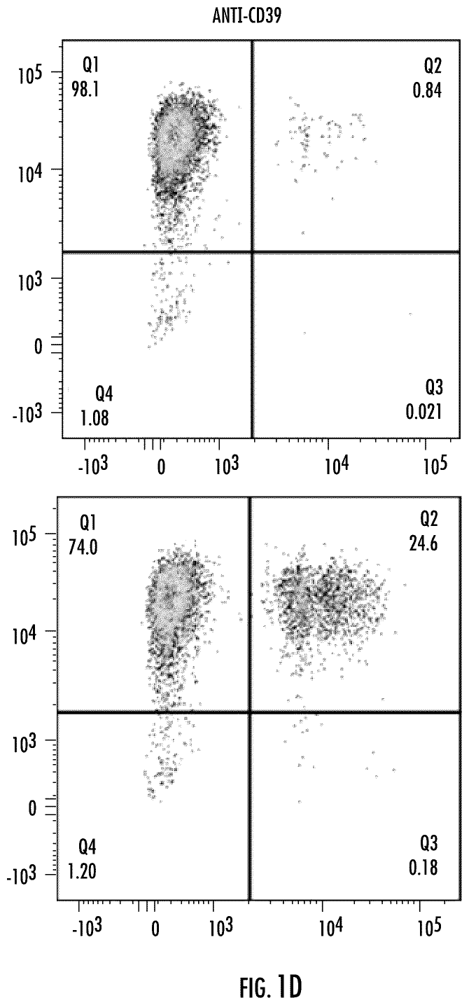

Fluorescent yellow and blue streptavidin polystyrene beads (1-1.5e8 particles) were coated with 25 ug of biotinylated chicken a-FPV antibodies, washed and incubated overnight at Room Temperature (RT) with FPV-H5-CD20-F or FPV-H-CD39-F at 5:1 beads to virus ratio. Beads were then washed and blocked with 1% BSA, 2% FBS PBS overnight a RT. Beads were resuspended in 100u1 of 1XPBS 1% BSA and used for staining. CHO stable cell lines expressing membrane bound human anti-CD20 (mab271) or anti-CD39 (mab26086) antibodies (2E5 cells) were blocked with 1×PBS 1% BSA 2% FBS for 1 h on ice, washed, treated with Heparin 100 Units in 1XPBS, 1% BSA for 20 min at RT, dispensed into flow staining tubes and incubated with 40 μl of yellow (FIG. 1A and B) or 25 μl of blue (FIG. 1C and D) FPV coated beads for 1 hour at RT with occasional mixing. Cells were then washed twice with 1XPBS 1% BSA and resuspended in secondary goat a-human Fab-APC or goat a-human Fab-FITC antibodies and incubated for 30 min at 4° C. Cells were pelleted, washed twice and fixed with 0.5% paraformaldehyde in 1% BSA PBS before running on the BD FACS Canto II with propidium iodide for live/dead discrimination. The cells expressing antibody and binding FPV coated beads were identified as double positive events. (FIG. 1A-D). Good signal to noise was observed using control antibody transfectants and two different fluorescent virus constructs.

### SEMA4D

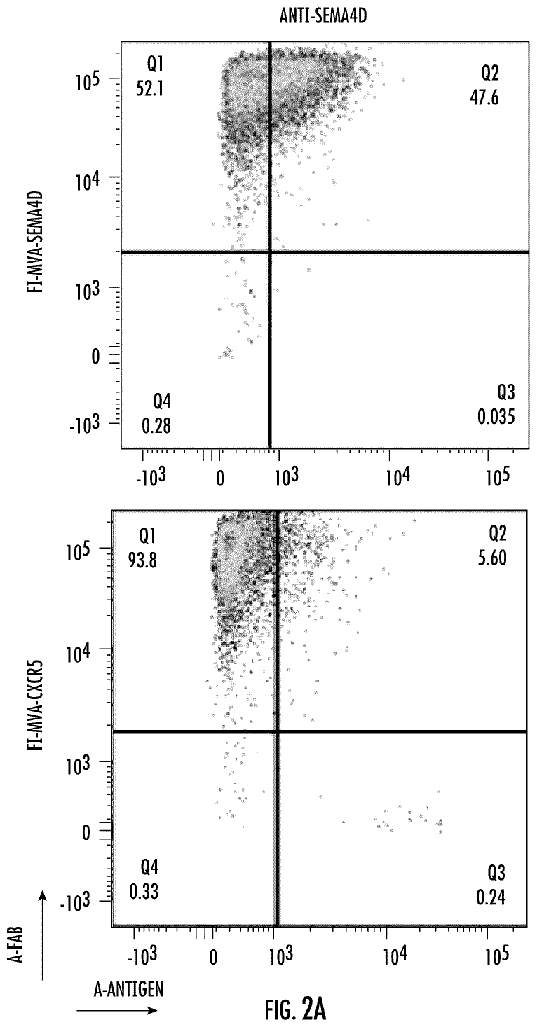

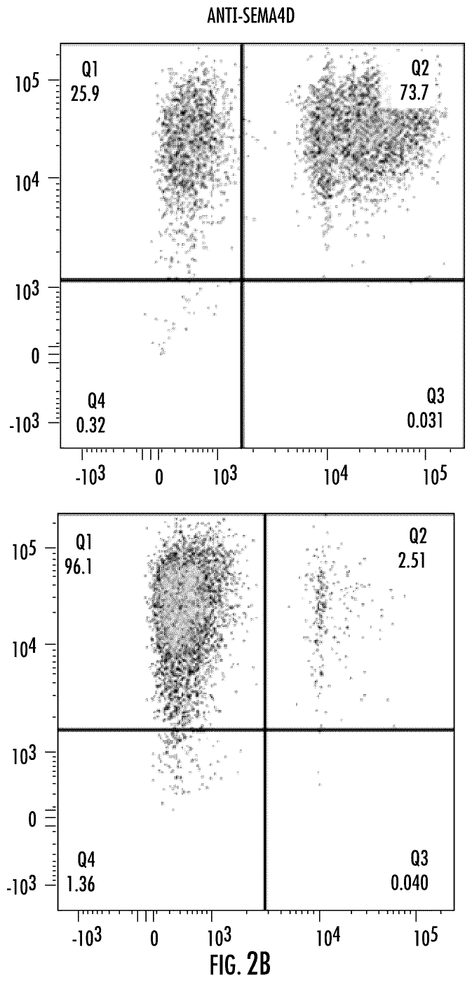

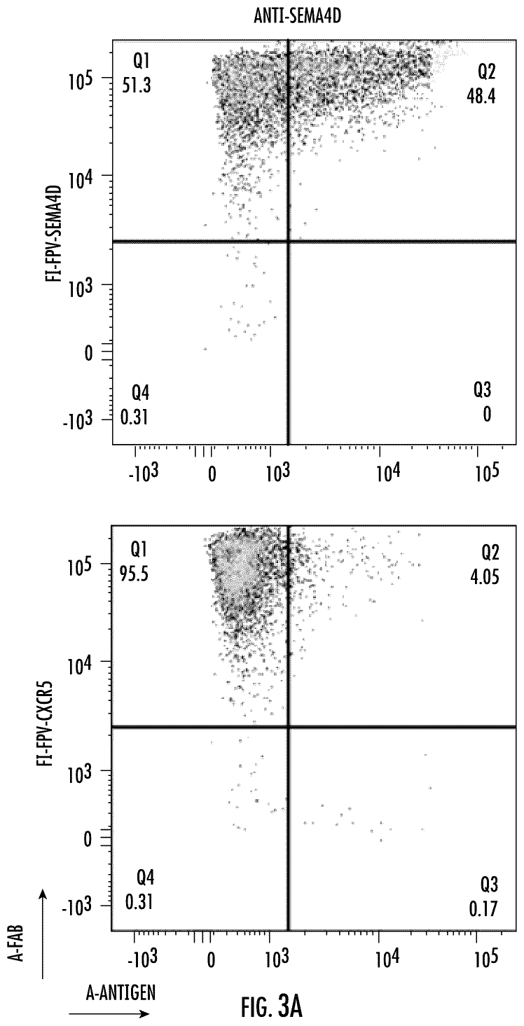

Fluorescent yellow and blue streptavidin polystyrene beads (1-1.5e8 particles) were incubated with FSL biotinylated MVA-SEMA-ECD and MVA-CXCR5-G-SL EEV at 5:1 beads-to-virus ratio overnight at RT in the dark. Beads were then washed and blocked with 1% BSA, 2% FBS PBS overnight at RT. Beads were resuspended in 100 μl of 1XPBS 1% BSA and used for staining. CHO stable cell lines expressing membrane bound human anti SEMA4D (mab67) antibody (2E5 cells) were blocked with 1×PBS 1% BSA 2% FBS for 1 hour on ice, washed, treated with Heparin 100u in 1XPBS, 1% BSA for 20 min at room temperature, dispensed into flow staining tubes and incubated with 40 μl of yellow (FIG. 2A, left panel) or 25 μl of blue (FIG. 2B, right panel) MVA coated beads for 1 hour at room temperature with occasional mixing. Cells were then washed twice with 1XPBS, 1% BSA and resuspended in secondary APC or FITC conjugated goat a-human Fab antibody and incubated for 30 min at 4° C. Cells were pelleted, washed twice and fixed with 0.5% paraformaldehyde in 1% BSA PBS before running on the BD FACS Canto II with propidium iodide for live/dead discrimination. The cells expressing antibody and binding MVA coated beads were identified as double positive events. (FIG. 2A-D). Good signal to noise was observed using control antibody transfectants and two different fluorescent virus constructs.

### SEMA4D and SEMA4D IgG Library

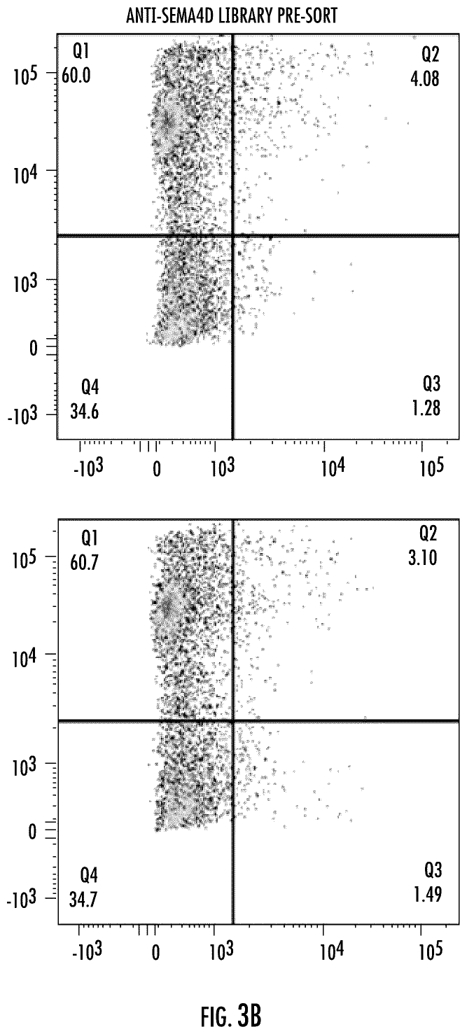

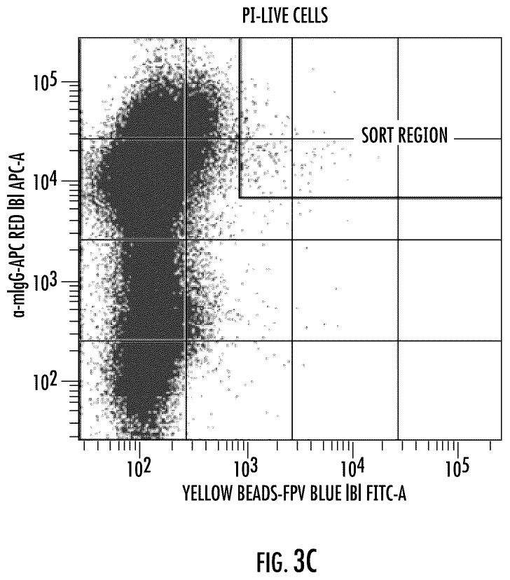

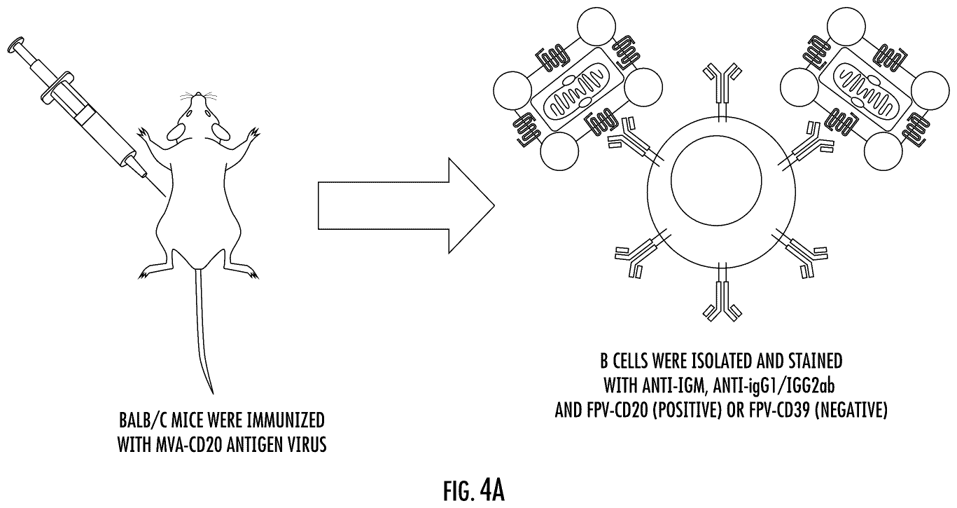

CHO cells stably expressing membrane bound human anti-SEMA4D (FIG. 3A), or SEMA4D IgG Library were blocked with 1×PBS 1% BSA 2% FBS for 1 h on ice, washed, treated with Heparin 100 Units in 1XPBS, 1% BSA for 20 min at room temperature, dispensed into flow staining tubes (2e5 cells/tube) and incubated with fluorescent yellow streptavidin beads coated with FPV-SEMA4D or FPV-CXCR5 for 1 hour at room temperature in the dark. For SEMA4D IgG Library sorting, 9e6 cells were resuspended in 3.6 ml of 1XPBS 1% BSA, dispensed into flow staining tubes (0.4 ml/tube) and incubated with fluorescent yellow streptavidin beads coated with FPV-SEMA4D (40u1/tube). Cells were then washed twice with 1XPBS 1% BSA, resuspended in secondary anti human Fab-APC and incubated for 30 min at 4° C. Cells were pelleted, and washed twice. For flow cytometry analysis and pre-sort evaluation, cells were fixed with 0.5% paraformaldehyde in 1% BSA PBS before running on the BD FACS Canto II with propidium iodide for live/dead discrimination. For SEMA4D IgG Library sorting, cells were resuspended in 1XPBS 1% BSA and filtered through a 40 μm filter. Sorted cells were pelleted and washed with 1×PBS. Cell pellets were kept at −20° C. before genomic DNA extraction and PCR. The results are shown in FIGS. 3A and B. As can be seen in FIG. 3C, recovery of anti-SEMA4D antibodies post-sort was 5% positive clones.

B cells sorted on B220+, IgM(−), IgG1(+) IgG2ab(+): FPV-CD20 and FPV-CD39

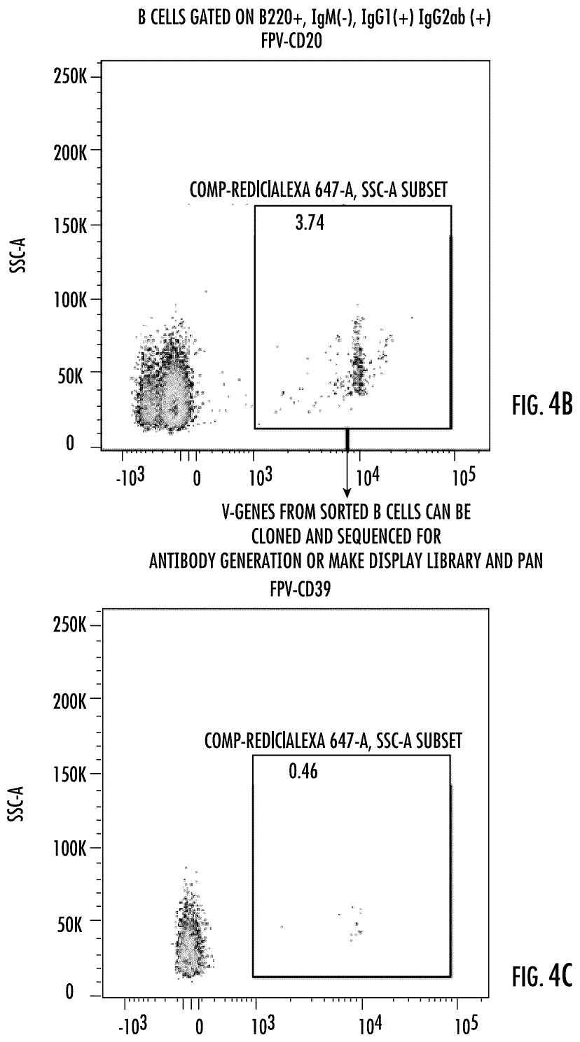

Balb/c mice were immunized three times with EEV MVA-T7-CD20-G-F. Six days after last immunization, mice were sacrificed and spleens were harvested for B cell isolation following the Miltenyi Biotech B cell negative selection protocol. B cells were blocked with 1% BSA, 2% FBS in 1×PBS, pH 7.2 for one hour on ice. B cells were next pelleted and resuspended in 0.4 ml of 1% BSA in 1x PBS, pH 7.2 per 1E7 cells. Fluorescent Blue beads coated with FPV-H5-CD20-F EEV or FPV-H5-CD39-F EEV (negative control) were added to the B cells (25 ul per 1e7 cells) and incubated 1 hour at Room Temperature in the dark with gentle mixing every 15 minutes. The B cells were then washed twice with 2 mL of 1% BSA in 1x PBS, pH7.2 and resuspended in 0.4 ml/1e7 cells of the same. Secondary antibodies were added: anti-B220-FITC, anti-mIgG1-BV421, anti-mIgG2a and 2b-BV421 and anti-mIgM-PerCP-eFluor 710 and incubated for 30 minutes at 4° C. B cells were washed twice with 2 mL 1% BSA in 1x PBS, pH 7.2 and resuspended in the same. The results are shown in FIG. 4A-D.

A phage display library was created from the sorted cells. The phage library was panned on FPV-CD20 for three rounds and then specific anti-CD20 antibodies were tested. New CD20 antibodies that were identified are shown in Table 1 below.

TABLE 1

| | | SEQ |
|---|---|---|
| | | ID |
| mAB | Sequence Heavy Chain (H)/Light Chain (L) | NO. |
| 15908m | H15760: | SEQ |
| | QVQLQQSGAELAKPGASVKMSCKASGYIFTRYWIHWVKQRPGQG | ID |
| | LEWIGYITPSTGYTDYNQRFKGKATLTADKSSGTAYMQLSSLTSE | NO: |
| | DSAVYYCARDTFDFWGQGTTVTVSS | 38 |
| | L15141: | SEQ |
| | QIVLTQSPAIMSASPGEKVTMTCSARSSISYMHWYQQKPGTSPKR | ID |
| | WIYDTSKLASGVPARFSGSGSGTSYSLTISSMEAEDAATYYCHQRS | NO: |
| | SYPYTFGSGTKLEIK | 39 |
| 15920m | H15767: | SEQ |
| | QVQLQQSGAELAKPGASVKMSCKASGYTFTTYWIHWVKQRPGQ | ID |
| | GLEWIGYITPSTGYTDYNQKFKGKATLTADRSSSTAYMQLSSLTSE | NO: |
| | DSAVYYCARDTFDYWGQGTAVTVSS | 40 |
| | L15148: | SEQ |
| | QIVLTQSPTTMAASPGEKITVTCSTSSSISSNYLHWFQQKPGFSPKL | ID |
| | LIYGTSNLASGVPARFSGSGSGTSYSLTIGTMEAEDVATYYCQQGT | NO: |
| | SIPFTFGSGTKLEIK | 41 |
| 15929m | H15771: | SEQ |
| | QVQLQQSGAELVKPGASVKMSCKASGYTFTSYNMHWVKQTPGQ | ID |
| | GLEWVGAIYPGNGDTSYNQKFKGKATLTADKSSSTAYMQLSSLT | NO: |
| | SEDSAVYYCARFDYGPYALDYWGQGTTVTVSS | 42 |
| | L15172: | SEQ |
| | QIVLTQSPAIMSASPGEKVTMTCSASSSVSYMHWYQQKSGTSPKR | ID |
| | WIYDTSKLASGVPARFSGSGSGTSYSLTISSMEAEDAATYYCQQW | NO: |
| | TFNPPTFGGGTKLEIK | 43 |
| 15930m | H15772: | SEQ |
| | QVQLQQSGAELAKPGASVRLSCKASGYTFTSYWMYWVKQRPGQ | ID |
| | GLEWIGYITPTTYYTDYNRKFKDKATLTADKSSNTAYMQLSSLTS | NO: |
| | EDSAVYYCVRGGYDHRGFAYWGQGTTVTVSS | 44 |
| | L15156: | SEQ |
| | DIVMTQSPAIMSASPGEKVTMTCSASSSVSYMYWYQQKPGSSPRL | ID |
| | LIYDTSNLASGVPVRFSGSGSGTSYSLTISRMEAEDAATYYCQQWS | NO: |
| | SYPFTFGSGTKLEIK | 45 |
| 15931m | H15773: | SEQ |
| | QVQLQQSGAELAKPGASVKMSCKASGYTFSSFWMHWFRQRPGQ | ID |
| | GLEWIGYITPTTGYTDYNQKFKGKATLTADKSSSTAYMQLSSLTS | NO: |
| | EDSAVYYCARHSDGYYSYWYFDVWGQGTTVTVSS | 46 |
| | L15178: | SEQ |
| | DIVMTQSQEFMSTSVGDRVSVTCKASQNVGTNVAWYQQKPGQSP | ID |
| | KLLIYSASYRYTGVPDRFTGSGSGTDFTLTISNVQSEDLAEYFCQQ | NO: |
| | YNSYPLFTFGSGTKLEIK | 47 |
| 15937m | H15778: | SEQ |
| | QVQLQQSGAELAKPGASVKMSCKTSGYTFTNYWMHWVKQRPG | ID |
| | QGLEWIGYITPTTGYTDYNRKFKDKVALTADKSSSTAYMHLSSLT | NO: |
| | SEDSAVYYCARTAATSSYTMDYWGQGTAVTVSS | 48 |
| | L15167: | SEQ |
| | DIVMTQSPAIMSASPGEKVTMTCSASSSVSYMYWFQQKPGSSPRL | ID |
| | LIYDTSNLASGVPIRFSGSGSGTSYSLTISRMEAEDAATYYCQQWS | NO: |
| | SYPLTFGAGTKLEIK | 49 |
| 15960m | H15782: | SEQ |
| | QVQLQQSGAELAKPGASVKMSCKASGYTFTDYWMYWAKQRPG | ID |
| | QGLEWIGYITPTTGYTDYNQKFKAKATLTADKSSSTAYMQLSSLT | NO: |
| | SEDSAVYYCGRTGLGRGIDHWGQGTTVTVSS | 50 |
| | L15179: | SEQ |
| | DIVMTQSPAIMSASPGEKVTMTCRASSSVSSSYLHWYQQKSGASP | ID |
| | KLWIYSTSNLASGVPARFSGSGSGTSYSLTISSVEAEDAATYYCQQ | NO: |
| | YSGYPLTFGAGTKLEIK | 51 |

### Example 6: Plasma Cell Isolation and ELISA for CXCR4

Mice were immunized four times with MVA-CXCR4 intracellular mature virus (IMV) intraperitoneally. On day 5 post final boost, the spleen and bone marrow were harvested into 1X PBS, pH 7.2, separately. The spleen was cut into small pieces and ground through a 40 uM filter. Spleen cells were washed 2 times with 1×PBS, pH 7.2. After removal of the PBS, red blood cells were lysed using Lysis buffer (Biolegend, cat #420301) as per the manufacturer's protocol. Separately, the bone marrow was filtered with a 40 uM filter and washed 2 times with 1x PBS, pH 7.2. Plasma cells were then isolated, from the bone marrow and spleen separately, using the CD138+Plasma cell isolation kit (Milentyi, cat #30-092-530) by following the manufacturer's protocol. Cells were counted and seeded at 1000 cells/well into V-bottom 96 well plates in plasma cell complete growth media (Growth media: RPMI, 10% FBS, 1X Beta-mercaptoethanol, 10 ng/ml CXCL12, 50 ng/ml APRIL, 5 ng/ml IL-6, 5 ng/ml BAFF, 5 ng/ml IL-4). After 72 hours, supernatant was harvested and the Plasma Cell ELISA was performed.

For the Plasma Cell ELISA, ELISA plates were coated at 2E6 pfu/ml with either FPV-CXCR4opt-GFP-F13L crude EEV or FPV wt crude EEV and incubated overnight at 4° C. The following day, plates were washed with 1×PBS pH 7.2, 0.05% Tween 20, three times, and blocked with 250u1/well of 1×PBS pH7.2, 1% BSA, for 1 hour at room temperature. Plates were washed three times again, and after diluting 1:2, 100u1 of supernatant was added to each virus coated plate. Plates were incubated for 1 hour at room temperature. After washing three times, 100 uL/well of 1:40,000 anti-mouse IgG (H+L)-Biotin (Jackson cat #115-065-166) was added to each well for detection. Plates were incubated for 1 hour at room temperature and then washed three times. Strepavidin-HRP (Thermo Fisher, cat #SNN2004), at 1:8000, was added to each well (100u1), and incubated for 30 minutes at room temperature. Plates were washed three times and 100u1 of TMB substrate (BioFx, cat #TMBW-1000-01) was added to develop the plates. After 15 minutes, 100u1 of 2N sulfuric acid was added to stop the development. Plates were read at 450-570 nM on the BioTek Power Wave reader.

After the readout, positive wells containing the plasma cells, were diluted to 100 cells/well in plasma cell complete growth media. After 72 hours, supernatant was collected and the plasma cell ELISA was performed as described above. Positive plasma cell wells were stored in 100u1 of RNALater (Invitrogen, cat #AM7021). Plasma cells were recovered used to generate a phage display antibody library.

### Example 7: Plasma Cell Isolation and ELISA for CD20

Mice were immunized three times with either MVA-CD20 IMV or EEV intraperitoneally. On day 4 after the last boost, the spleen and bone marrow were harvested into 1x PBS, pH 7.2, separately. The bone marrow was processed, similarly, as for the previous example, with the exception that the plasma cells were diluted out after isolation into 100cells/well.

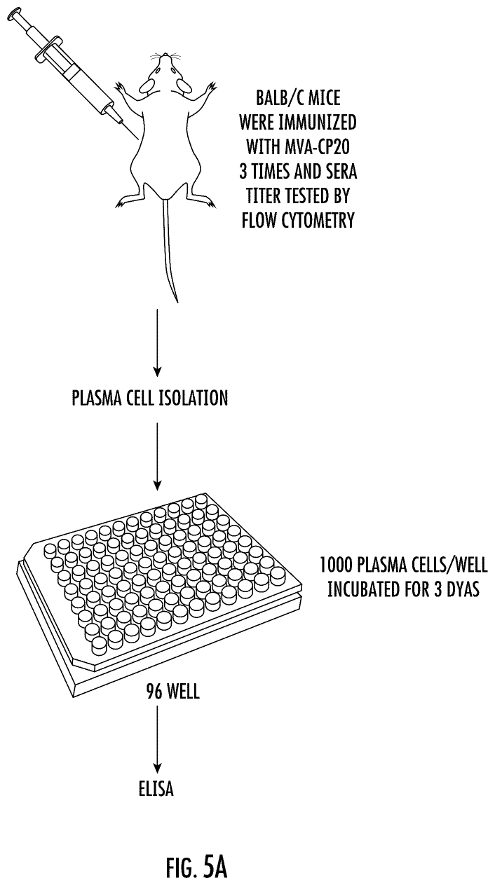

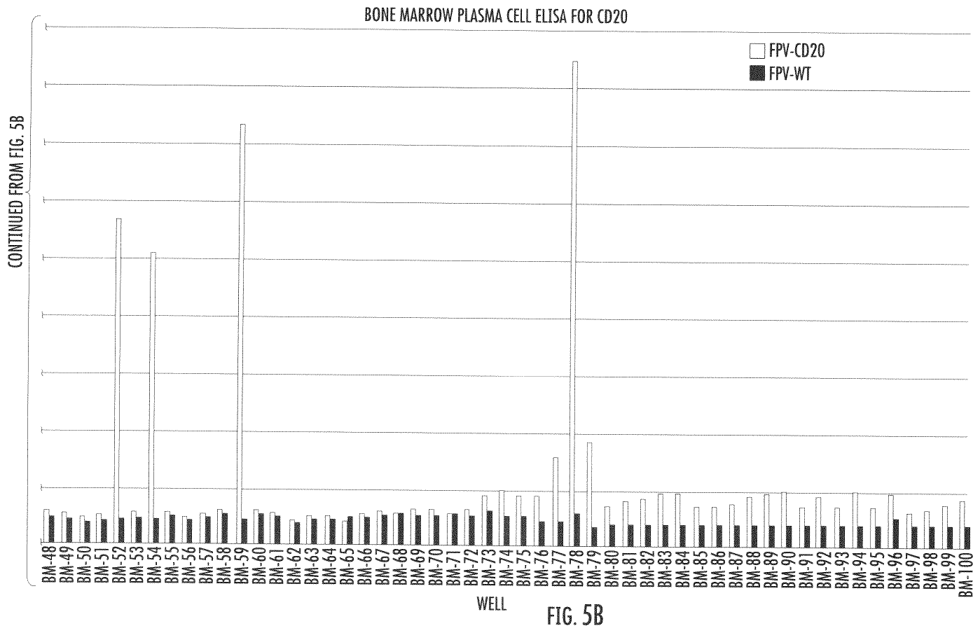

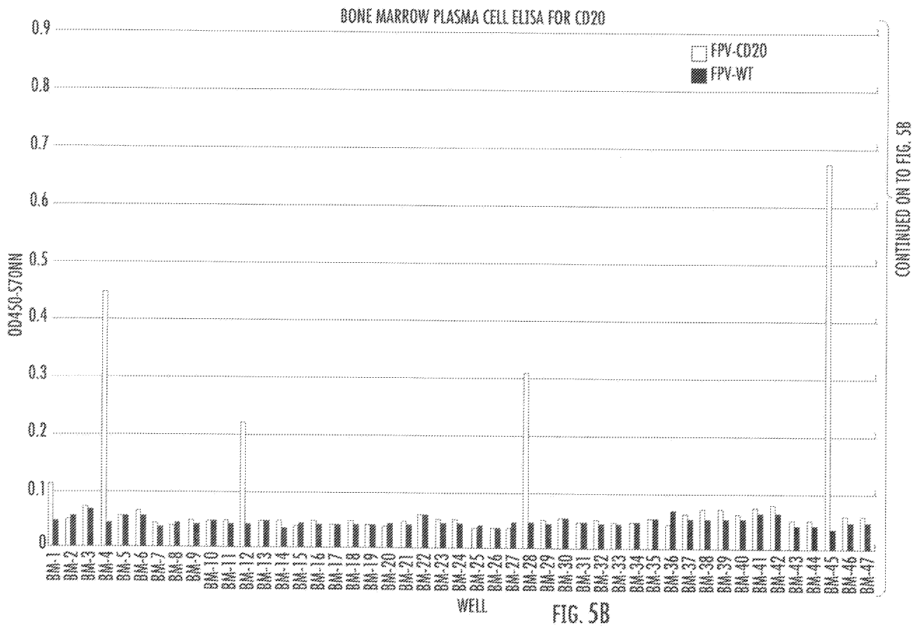

For the Plasma Cell ELISA, ELISA plates were coated at 2E6 pfu/ml with either FPV-CD20-F13L crude EEV or FPV wt crude EEV and incubated overnight at 4° C. The following day, the ELISA was performed as indicated in the previous example. (See FIG. 5A for strategy).

After the readout, RNALater (Invitrogen, cat #AM7021) was added to positive wells containing the plasma cells. Plasma cells that were recovered were used to generate an antibody display library. The phage display library was then panned on FPV CD20 and specific anti-CD20 antibodies were tested. New CD20 antibodies that were identified are shown in Table 2 below. Individual histograms of the antibodies are shown in FIG. 5B.

TABLE 2

| | | SEQ |
|---|---|---|
| | | ID |
| mAB | Sequence Heavy Chain (H)/Light Chain (L) | NO. |
| 15965m | H15785: | SEQ |
| | QVQLQQSGAELAKPGASVKMSCKTSGYTFTSYWMHWVKERPGQ | ID |
| | GLEWIGYITPSTGYTDYNQKFKDKATLTADKSSSTAYMQLISLTSE | NO: |
| | DSAVYYCARDGYPYAMDYWGQGTTVTVSS | 52 |
| | L15197: | SEQ |
| | DIVMTQSPAIMSASPGEKVTMTCRASSSVSSSYLHWYQQKSGASP | ID |
| | KLWIYSTSNLASGVPARFSGSGSGTSYSLTISSVEAEDAATYYCQQ | NO: |
| | YSGYPLRTFGGGTKLEIK | 53 |
| 15970m | H15789: | SEQ |
| | EVQLQESGAELAKPGASVKMSCKASGYTFTTYWMHWVKQRPGQ | ID |
| | GLEWIGYITPSTGYTDYNQKFKDKATLTADKSSSTAYMQLSSLTS | NO: |
| | EDSAVYYCARNYYGSGYAVDYWGQGTTVTVSS | 54 |
| | L15223: | SEQ |
| | DVVMTQSQKFMSTSVGDRVSITCKASQNVGAAVAWYQQKPGQS | ID |
| | PKLLIYSASSRYTGVPGRFTGSGSGTDFTLTISNMQSEDLADYFCQ | NO: |
| | QYSSYPLTFGGGTKLEIK | 55 |
| 15998m | H15799: | SEQ |
| | QVQLQQSGAELAKPGASVKMSCKASGYTFTTYWMHWVKQRPG | ID |
| | QGLEWIGYITPSTGYTDYNQKFKDKATLTADKSSSTAYMQLSSLT | NO: |
| | SEDSAVYYCARNYYGSGYAVDYWGQGTTVTVSS | 56 |
| | L15329: | SEQ |
| | DVVMTQSRKFMSTSVGDRVSITCKASQNVGAAVAWYQQKPGQS | ID |
| | PKLLIYSASSRYTGVPDRFTGSGSGTDFTLTISNMQSEDLADYFCQ | NO: |
| | QYSSYPQTFGAGTKLEIK | 57 |
| 16034m | H15808: | SEQ |
| | QVQLQQSGADLVKPGASVKMSCKASGYTFTSYNMHWVKQTPGQ | ID |
| | GLEWIGAIYPGNGDTSYNQKFKGKATLTADKSSSTAYMQLSSLTS | NO: |
| | ADSAVYYCARVMISTRGYWYFDVWGQGTTVTVSS | 58 |
| | L15192: | SEQ |
| | DIVMTQSPAILSASPGEKVTMTCRASSSVSYMHWYQQKPGSSPKP | ID |
| | WIYATSNLASGVPARFSGSGSGTSYSLTISRVEAEDAATYYCQQW | NO: |
| | GSNPPTFGSGTKLEIK | 59 |
| 16038m | H15809: | SEQ |
| | QVQLQQSGAELVKPGASVKMSCKASGFTFTSYNLHWVKQTPGQG | ID |
| | LEWIGGIYPGNGDTSYNQKFKGKATLTADKSSSTAYMQLSALTSE | NO: |
| | DSAVYYCARWAYYGNSGALDYWGQGTTVTVSS | 60 |
| | L15192: | SEQ |
| | DIVMTQSPAILSASPGEKVTMTCRASSSVSYMHWYQQKPGSSPKP | ID |
| | WIYATSNLASGVPARFSGSGSGTSYSLTISRVEAEDAATYYCQQW | NO: |
| | GSNPPTFGSGTKLEIK | 59 |

What is claimed is:

1. A method to select binding molecules that bind to a target integral membrane protein (IMP) comprising:
(a) attaching an antigen virion comprising a poxvirus that comprises the integral membrane protein (IMP) or a fragment thereof fused with an extracellular enveloped virion (EEV) protein or a functional fragment thereof to a solid support to form a coupled antigen virion, wherein the poxvirus expresses the target IMP or fragment thereof in native conformation as part of the outer envelope membrane of the EEV and wherein said IMP or fragment thereof comprises at least one extra-membrane region, at least one transmembrane region, and at least one intra-membrane region;
(b) contacting said coupled antigen virion with an antibody display library, wherein the library comprises display packages displaying a plurality of antigen binding domains;
(c) extracting antibody variable genes or fragments thereof from display packages that bind to the coupled antigen virion and cloning the variable light chain (VL) gene or fragment thereof and variable heavy chain (VH) gene or fragment thereof from said display package into a plasmid vector in frame with a polynucleotide sequence encoding a pox virus anchor protein or fragment thereof such that the VL, VH and sequence encoding the poxvirus anchor protein are co-expressed as a single polypeptide;
(d) transfecting mammalian cells with the plasmid vector of step (c) such that transfected cells express VL and VH antigen binding domains on the mammalian cell surface;
(e) screening the transfected cells with the antigen virion coupled to a detectable solid support; and
(f) recovering cells that display an antigen binding domain specific for the target.

2. The method of claim 1, wherein the solid support in step (a) is streptavidin labelled magnetic beads.

3. The method of claim 2, wherein the poxvirus is fowlpox virus labelled with a biotin-anti-fowlpox antibody.

4. The method of claim 2, wherein the poxvirus is a biotinylated vaccinia virus Ankara (MVA).

5. The method of claim 1, wherein the poxvirus anchor protein is the vaccinia virus A56R protein.

6. The method of claim 1, wherein the mammalian cells are CHO cells.

7. The method of claim 1, wherein the IMP is a multi-pass protein.

8. The method of claim 7, wherein the IMP is an ion channel or a G protein.

9. A method to select binding molecules that bind to a target integral membrane protein (IMP) comprising:
(a) isolating plasma cells from an animal immunized with an antigen comprising the target integral membrane protein (IMP) or fragment thereof;
(b) seeding the plasma cells into pools comprising a plurality of cells and growing them in nutrient media to a desired cell density;
(d) performing one or more assays of the plasma cells to identify cells that express binding molecules that bind to the target IMP protein; and
(e) recovering the plasma cells that express binding molecules that bind to the target IMP protein.

10. The method of claim 9, wherein a first ELISA is performed to identify cells that express binding molecules that bind to the target IMP protein, followed by at least one further ELISA assay in which the cells that are identified by the first ELISA assay are diluted prior to performing the further ELISA assay to identify cells that express binding molecules that bind to the target IMP protein.

11. The method of claim 9, wherein the cells that are recovered are used to generate an antibody display library, wherein the library comprises display packages displaying a plurality of antigen binding domains.

12. The method of claim 9, wherein the IMP is a multi-pass protein.

13. The method of claim 9, wherein the mammal is a mouse.

14. A method to select binding molecules that specifically bind to a target integral membrane protein (IMP) comprising:
(a) isolating B cells from an animal immunized with the target integral membrane protein (IMP) or fragment thereof;
(b) sorting the B cells to isolate antigen-specific B cells that express IgG that specifically binds target IMP; and
(c) performing single cell analysis to identify the Immunoglobulin variable region genes expressed by the sorted B cells.

15. The method of claim 14, wherein a phage Fab display library is generated from variable heavy chain (VH) and variable light chain (VL) cDNAs generated from RNA isolated from the antigen-specific B cells that express IgG that binds target IMP; and the phage Fab display library is panned to eliminate anti-poxvirus binding molecules and enrich for anti-target IMP binding molecules.

16. The method of claim 14, further comprising isolating and cloning the variable heavy chain genes and/or variable light chain genes from individual sorted B cells.

17. The method of claim 14, wherein the single cell analysis comprises RT-PCR.

18. The method of claim 14, wherein the B cells are sorted with the target IMP coupled to a detectable solid support.

19. The method of claim 18, wherein the detectable solid support is a streptavidin-fluorescent bead.

20. The method of claim 15, further comprising isolating the VH and VL genes (V genes) from the phage Fab display library and subcloning the V genes into a mammalian expression vector while maintaining VH and VL pairing that was present in individual phage as a mini-library (ML).

21. The library of claim 15.

22. An antibody that specifically binds to CD20, wherein said antibody is selected from the group consisting of antibodies comprising a Variable Heavy chain (VH) of SEQ ID NO: 38 and Variable Light chain (VL) of SEQ ID NO: 39; a VH of SEQ ID NO: 40 and a VL of SEQ ID NO: 41; a VH of SEQ ID NO: 42 and a VL of SEQ ID NO: 43; a VH of SEQ ID NO: 44 and a VL of SEQ ID NO: 45; a VH of SEQ ID NO: 46 and a VL of SEQ ID NO: 47; a VH of SEQ ID NO: 48 and a VL of SEQ ID NO: 49; a VH of SEQ ID NO: 50 and a VL of SEQ ID NO: 51; a VH of SEQ ID NO: 52 and a VL of SEQ ID NO: 53; a VH of SEQ ID NO: 54 and a VL of SEQ ID NO: 55; a VH of SEQ ID NO: 56 and a VL of SEQ ID NO. 57; a VH of SEQ ID NO: 58 and a VL of SEQ ID NO: 59; and a VH of SEQ ID NO: 60 and a VL of SEQ ID NO: 59.


SEQUENCE LISTING (60 sequences)

SEQ ID NO: 1 - protein, 314 aa, Vaccinia virus
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 2 - protein, 237 aa, synthetic construct
MGWSCIILFLVATATGVHSDIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 674 aa, synthetic construct
MGWSCIILFLVATATGAHSQVQLQQSGPELVKPGASVKISCKASGYSFSDYYMHWVKQSPENSLEWIGQINPTTGGASYNQKFKGKATLTVDKSSSTAYMQLKSLTSEESAVYYCTRYYYGRHFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 4 - protein, 232 aa, synthetic construct
MGWSCIILFLVATATGVHSQIVLTQSPAIMSASPGEKVTMTCSASSSVYYMHWYQQKSGTSPKRWIYDTSKLASGVPARFSGSGSGTSYSLTISSMEAEDAATYYCQQWSSNPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 675 aa, synthetic construct
MGWSCIILFLVATATGAHSQVQLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARSGTGSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 6 - protein, 232 aa, synthetic construct
MGWSCIILFLVATATGVHSSYELTQPPSVSVSPGQTASITCSGDKLGDKYASWYQQKPGQSPVLVIYQDSKRPSGIPERFSGSNSGNTATLTISGTQAMDEADYYCQAWDSSTAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 679 aa, synthetic construct
MGWSCIILFLVATATGAHSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRYSRYYDYLGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 8 - DNA, 30 bp, synthetic construct
  source 1..30: mol_type = other DNA
gatggagctgtatcatcctcttcttggtag

SEQ ID NO: 9 - DNA, 24 bp, synthetic construct
  source 1..24: mol_type = other DNA
tggtgttgctgggcttgtgattca

SEQ ID NO: 10 - DNA, 25 bp, synthetic construct
  source 1..25: mol_type = other DNA
agttaggccagcttggcacttgatg

SEQ ID NO: 11 - DNA, 25 bp, synthetic construct
  source 1..25: mol_type = other DNA
actgtcactggctcagggaaatagc

SEQ ID NO: 12 - DNA, 24 bp, synthetic construct
  source 1..24: mol_type = other DNA
aggagccagttgtatctccacaca

SEQ ID NO: 13 - DNA, 20 bp, synthetic construct
  source 1..20: mol_type = other DNA
tggaatttgccctttttgag

SEQ ID NO: 14 - DNA, 23 bp, synthetic construct
  source 1..23: mol_type = other DNA
aagtagtccttgaccaggcagcc

SEQ ID NO: 15 - protein, 11 aa, synthetic construct
ECMVIRESFWD

SEQ ID NO: 16 - DNA, 18 bp, synthetic construct
  source 1..18: mol_type = other DNA
tacatgtgtttagtcgag

SEQ ID NO: 17 - DNA, 25 bp, synthetic construct
  source 1..25: mol_type = other DNA
ggagttagtttgggcagcagatcca

SEQ ID NO: 18 - DNA, 21 bp, synthetic construct
  source 1..21: mol_type = other DNA
tccagagttccaggtcaaggt

SEQ ID NO: 19 - DNA, 37 bp, synthetic construct
  source 1..37: mol_type = other DNA
aatatgcgcgcactcccaggtccagctgcagcagtct

SEQ ID NO: 20 - DNA, 36 bp, synthetic construct
  source 1..36: mol_type = other DNA
aatatgcgcgcactcccaggtgcagctgaaggagtc

SEQ ID NO: 21 - DNA, 34 bp, synthetic construct
  source 1..34: mol_type = other DNA
aatatgcgcgcactccgaggtgcagcttcaggag

SEQ ID NO: 22 - DNA, 38 bp, synthetic construct
  source 1..38: mol_type = other DNA
aatatgcgcgcactccgaggtgaagcttctcgagtctg

SEQ ID NO: 23 - DNA, 38 bp, synthetic construct
  source 1..38: mol_type = other DNA
aatatgcgcgcactccgaagtgaagctggtggagtctg

SEQ ID NO: 24 - DNA, 29 bp, synthetic construct
  source 1..29: mol_type = other DNA
gagacggtgaccgtggtcccttggcccca

SEQ ID NO: 25 - DNA, 26 bp, synthetic construct
  source 1..26: mol_type = other DNA
tgctcactggatggtgggaagatgga

SEQ ID NO: 26 - DNA, 33 bp, synthetic construct
  source 1..33: mol_type = other DNA
ccgtttgatctcgagcttggtgcctccaccgaa

SEQ ID NO: 27 - DNA, 33 bp, synthetic construct
  source 1..33: mol_type = other DNA
ccgttttatctcgagcttggtcccccctccgaa

SEQ ID NO: 28 - DNA, 33 bp, synthetic construct
  source 1..33: mol_type = other DNA
ccgttttatctcgagtctggtcccatcactgaa

SEQ ID NO: 29 - DNA, 33 bp, synthetic construct
  source 1..33: mol_type = other DNA
ccgttttatctcgagctttgtccccgagccgaa

SEQ ID NO: 30 - DNA, 33 bp, synthetic construct
  source 1..33: mol_type = other DNA
ccgtttcagctcgagcttggtcccagcaccgaa

SEQ ID NO: 31 - DNA, 37 bp, synthetic construct
  source 1..37: mol_type = other DNA
gctatagtgcactccgacattgtgatgacccagtctc

SEQ ID NO: 32 - DNA, 35 bp, synthetic construct
  source 1..35: mol_type = other DNA
gctatagtgcactccgacattgtgatgacacagtc

SEQ ID NO: 33 - DNA, 32 bp, synthetic construct
  source 1..32: mol_type = other DNA
gctatagtgcactccgatgttgtgatgaccca

SEQ ID NO: 34 - DNA, 34 bp, synthetic construct
  source 1..34: mol_type = other DNA
gctatagtgcactccgacattgtgctgactcagt

SEQ ID NO: 35 - DNA, 37 bp, synthetic construct
  source 1..37: mol_type = other DNA
gctatagtgcactccgacattgtgctgacccaatctc

SEQ ID NO: 36 - DNA, 37 bp, synthetic construct
  source 1..37: mol_type = other DNA
gctatagtgcactcccaaattgttctcacccagtctc

SEQ ID NO: 37 - DNA, 36 bp, synthetic construct
  source 1..36: mol_type = other DNA
gctatagtgcactccgacatccagatgacccagtct

SEQ ID NO: 38 - protein, 114 aa, synthetic construct
QVQLQQSGAELAKPGASVKMSCKASGYIFTRYWIHWVKQRPGQGLEWIGYITPSTGYTDYNQRFKGKATLTADKSSGTAYMQLSSLTSEDSAVYYCARDTFDFWGQGTTVTVSS

SEQ ID NO: 39 - protein, 106 aa, synthetic construct
QIVLTQSPAIMSASPGEKVTMTCSARSSISYMHWYQQKPGTSPKRWIYDTSKLASGVPARFSGSGSGTSYSLTISSMEAEDAATYYCHQRSSYPYTFGSGTKLEIK

SEQ ID NO: 40 - protein, 114 aa, synthetic construct
QVQLQQSGAELAKPGASVKMSCKASGYTFTTYWIHWVKQRPGQGLEWIGYITPSTGYTDYNQKFKGKATLTADRSSSTAYMQLSSLTSEDSAVYYCARDTFDYWGQGTAVTVSS

SEQ ID NO: 41 - protein, 108 aa, synthetic construct
QIVLTQSPTTMAASPGEKITVTCSTSSSISSNYLHWFQQKPGFSPKLLIYGTSNLASGVPARFSGSGSGTSYSLTIGTMEAEDVATYYCQQGTSIPFTFGSGTKLEIK

SEQ ID NO: 42 - protein, 119 aa, synthetic construct
QVQLQQSGAELVKPGASVKMSCKASGYTFTSYNMHWVKQTPGQGLEWVGAIYPGNGDTSYNQKFKGKATLTADKSSSTAYMQLSSLTSEDSAVYYCARFDYGPYALDYWGQGTTVTVSS

SEQ ID NO: 43 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWTFNPPTFGGGTKLEIK

SEQ ID NO: 44 - protein, 119 aa, synthetic construct
QVQLQQSGAELAKPGASVRLSCKASGYTFTSYWMYWVKQRPGQGLEWIGYITPTTYYTDYNRKFKDKATLTADKSSNTAYMQLSSLTSEDSAVYYCVRGGYDHRGFAYWGQGTTVTVSS

SEQ ID NO: 45 - protein, 106 aa, synthetic construct
DIVMTQSPAIMSASPGEKVTMTCSASSSVSYMYWYQQKPGSSPRLLIYDTSNLASGVPVRFSGSGSGTSYSLTISRMEAEDAATYYCQQWSSYPFTFGSGTKLEIK

SEQ ID NO: 46 - protein, 122 aa, synthetic construct
QVQLQQSGAELAKPGASVKMSCKASGYTFSSFWMHWFRQRPGQGLEWIGYITPTTGYTDYNQKFKGKATLTADKSSSTAYMQLSSLTSEDSAVYYCARHSDGYYSYWYFDVWGQGTTVTVSS

SEQ ID NO: 47 - protein, 108 aa, synthetic construct
DIVMTQSQEFMSTSVGDRVSVTCKASQNVGTNVAWYQQKPGQSPKLLIYSASYRYTGVPDRFTGSGSGTDFTLTISNVQSEDLAEYFCQQYNSYPLFTFGSGTKLEIK

SEQ ID NO: 48 - protein, 120 aa, synthetic construct
QVQLQQSGAELAKPGASVKMSCKTSGYTFTNYWMHWVKQRPGQGLEWIGYITPTTGYTDYNRKFKDKVALTADKSSSTAYMHLSSLTSEDSAVYYCARTAATSSYTMDYWGQGTAVTVSS

SEQ ID NO: 49 - protein, 106 aa, synthetic construct
DIVMTQSPAIMSASPGEKVTMTCSASSSVSYMYWFQQKPGSSPRLLIYDTSNLASGVPIRFSGSGSGTSYSLTISRMEAEDAATYYCQQWSSYPLTFGAGTKLEIK

SEQ ID NO: 50 - protein, 118 aa, synthetic construct
QVQLQQSGAELAKPGASVKMSCKASGYTFTDYWMYWAKQRPGQGLEWIGYITPTTGYTDYNQKFKAKATLTADKSSSTAYMQLSSLTSEDSAVYYCGRTGLGRGIDHWGQGTTVTVSS

SEQ ID NO: 51 - protein, 108 aa, synthetic construct
DIVMTQSPAIMSASPGEKVTMTCRASSSVSSSYLHWYQQKSGASPKLWIYSTSNLASGVPARFSGSGSGTSYSLTISSVEAEDAATYYCQQYSGYPLTFGAGTKLEIK

SEQ ID NO: 52 - protein, 118 aa, synthetic construct
QVQLQQSGAELAKPGASVKMSCKTSGYTFTSYWMHWVKERPGQGLEWIGYITPSTGYTDYNQKFKDKATLTADKSSSTAYMQLISLTSEDSAVYYCARDGYPYAMDYWGQGTTVTVSS

SEQ ID NO: 53 - protein, 109 aa, synthetic construct
DIVMTQSPAIMSASPGEKVTMTCRASSSVSSSYLHWYQQKSGASPKLWIYSTSNLASGVPARFSGSGSGTSYSLTISSVEAEDAATYYCQQYSGYPLRTFGGGTKLEIK

SEQ ID NO: 54 - protein, 120 aa, synthetic construct
EVQLQESGAELAKPGASVKMSCKASGYTFTTYWMHWVKQRPGQGLEWIGYITPSTGYTDYNQKFKDKATLTADKSSSTAYMQLSSLTSEDSAVYYCARNYYGSGYAVDYWGQGTTVTVSS

SEQ ID NO: 55 - protein, 107 aa, synthetic construct
DVVMTQSQKFMSTSVGDRVSITCKASQNVGAAVAWYQQKPGQSPKLLIYSASSRYTGVPGRFTGSGSGTDFTLTISNMQSEDLADYFCQQYSSYPLTFGGGTKLEIK

SEQ ID NO: 56 - protein, 120 aa, synthetic construct
QVQLQQSGAELAKPGASVKMSCKASGYTFTTYWMHWVKQRPGQGLEWIGYITPSTGYTDYNQKFKDKATLTADKSSSTAYMQLSSLTSEDSAVYYCARNYYGSGYAVDYWGQGTTVTVSS

SEQ ID NO: 57 - protein, 107 aa, synthetic construct
DVVMTQSRKFMSTSVGDRVSITCKASQNVGAAVAWYQQKPGQSPKLLIYSASSRYTGVPDRFTGSGSGTDFTLTISNMQSEDLADYFCQQYSSYPQTFGAGTKLEIK

SEQ ID NO: 58 - protein, 122 aa, synthetic construct
QVQLQQSGADLVKPGASVKMSCKASGYTFTSYNMHWVKQTPGQGLEWIGAIYPGNGDTSYNQKFKGKATLTADKSSSTAYMQLSSLTSADSAVYYCARVMISTRGYWYFDVWGQGTTVTVSS

SEQ ID NO: 59 - protein, 106 aa, synthetic construct
DIVMTQSPAILSASPGEKVTMTCRASSSVSYMHWYQQKPGSSPKPWIYATSNLASGVPARFSGSGSGTSYSLTISRVEAEDAATYYCQQWGSNPPTFGSGTKLEIK

SEQ ID NO: 60 - protein, 121 aa, synthetic construct
QVQLQQSGAELVKPGASVKMSCKASGFTFTSYNLHWVKQTPGQGLEWIGGIYPGNGDTSYNQKFKGKATLTADKSSSTAYMQLSALTSEDSAVYYCARWAYYGNSGALDYWGQGTTVTVSS